(19) United States
(12) Patent Application Publication
(10) Pub. No.: US 2022/0273733 A1
(43) Pub. Date: Sep. 1, 2022

(54) NUTRITIVE COMPOSITIONS WITH BIOACTIVE PROTEINS

(71) Applicant: Evolve BioSystems, Inc., Davis, CA (US)

(72) Inventors: Steven FRESE, Davis, CA (US); Samara FREEMAN-SHARKEY, Davis, CA (US); David KYLE, Davis, CA (US); Sercan KARAV, Canakkale (TR); Rebbeca M. DUAR, Vacaville, CA (US)

(21) Appl. No.: 17/629,865

(22) PCT Filed: Jul. 27, 2020
(86) PCT No.: PCT/US2020/043793

§ 371 Date: Jan. 25, 2022

Related U.S. Application Data
(60) Provisional application No. 62/879,298, filed on Jul. 26, 2019.

(51) Int. Cl. A61K 35/745; A61K 31/716; A61K 31/702; A61K 38/48; A61K 45/06; A61P 1/14; A61P 31/04

(52) U.S. Cl. CPC A61K 35/745; A61K 31/716; A61K 31/702; A61K 38/4826; A61K 45/06; A61P 1/14; A61P 31/04; C12Y 302/01096; C12Y 304/21001; C12Y 304/21004

(57) ABSTRACT

The inventions described herein relate generally to compositions comprising bioactive proteins including, but not limited to, enzymes and antimicrobial proteins. Such bioactive protein compositions may be present alone or in a mammalian milk or soy-based nutritional product to increase colonization of desired commensal organisms, reduce potential pathogens, restore microbiome function, and/or otherwise improve health in a mammal consuming same.

### FIELD OF INVENTION

The inventions described herein relate generally to compositions comprising bioactive proteins including, but not limited to, enzymes and antimicrobial proteins. Such bioactive protein compositions may be present alone or in a mammalian milk or soy-based nutritional product to increase colonization of desired commensal organisms, reduce potential pathogens, restore microbiome function, and/or otherwise improve health in a mammal consuming same. The compositions deliver additional functionality to the gastrointestinal tract through synergy with infant formula, and/or provide post-surgery, post-antibiotics, and/or post-fecal transplant recovery products that may be in a powdered or aqueous form in order to promote healthy function of the gut microbiome. They may be added directly or added as a supplement to the source of primary nutrition. Specifically, the inventions involve the purification of known, or novel, bioactive proteins and/or oligosaccharides, and novel combinations of these for use to stimulate microbiome function. Some iterations of this invention could include the addition of probiotics such as, but not limited to Bifidobacterium infantis, activated or otherwise, to a subject in need of reducing gut dysbiosis and/or promoting health.

### BACKGROUND

Beginning in infancy, the gut microbiome has been found to play an integral role in mammalian health. In human infants the composition of a healthy, or high functioning, gut microbiome consists of a monoculture, or nearly so, of Bifidobacterium longum subsp. infantis. Modern medical techniques like formula-feeding, antibiotics, and cesarean sections have had a disruptive effect on the infant gut microbiome leading to an unhealthy complexity of the microbiome. Infants expressing a complex microbiome composition are at an increased risk of health problems including infection by bacterial pathogens.

The mammalian gut microbiome is entirely dependent on the host organism for nutritional requirements and the composition of the microbiome will adapt in accordance with nutritional availability. Human milk oligosaccharides (“HMOs”) are an important component of breast milk and are required by B. infantis to establish a microbiome with B. infantis as the predominant species. Infant formula contains levels of oligosaccharides lower and less comprehensive than is required to establish a B. infantis predominant microbiome. Many bioactive proteins—those which have a health effect beyond nutritional value—are not present in formula and may contribute to creating a niche for B. infantis in dominating the microbiome. The inventors have discovered compositions of oligosaccharides and bioactive proteins which, when used as a supplement to infant formula, post-surgery recovery drink, or equivalent improves gut colonization of B. infantis.

### SUMMARY OF INVENTION

The inventors have discovered compositions and methods comprising the use of a functional bioactive protein in conjunction with a glycan and/or a Bifidobacterium strain added to the diet or as a therapeutic solution to an individual in need of reducing dysbiosis and/or improving intestinal function.

The composition described herein contains one or more functional bioactive proteins. These functional bioactive proteins may be either native or recombinant, and may comprise enzymes, glycoproteins, or glycopeptides. In some embodiments of this invention the functional bioactive protein is an enzyme, where the enzyme is a protease, lipase, amylase, lysozyme or endo-β-N-acetylglucosaminidase (EndoBI-1). Proteases may include, but are not limited to, trypsin, chymotrypsin, or homologues thereof. In any embodiment of the herein disclosed invention the functional bioactive protein component may comprise a natural and/or a recombinant protein or proteins. In other embodiments, the functional bioactive protein may be a glycoprotein, such as but not limited to lactoferrin. A glycopeptide may be exemplified by lactoferrin. The glycoprotein can be from an animal, plant, bacterial, or fungal source. The animal source may be milk, meat, eggs, egg whites, insects, fish, or from a culture of cells derived thereof. The plant source may be soy, sorghum, seeds, corn, peas, legumes, pulses, grains such as wheat, or others. In some embodiments of this invention the functional bioactive protein is lysozyme. Functional bioactive proteins discussed herein, recombinant or natural, may be purified and/or dried for addition to different compositions or for independent use.

Some embodiments of the herein disclosed invention comprise a combination of lysozyme and lactoferrin as the functional bioactive protein component. In some embodiments of the herein disclosed invention the lysozyme is present in a concentration of less than 0.1 g/L, 01.-1.5 g/L, 1.5 g/L-3.1 g/L, or 3.1 g/L or greater. In some embodiments of the herein disclosed invention the lactoferrin is present in a concentration of 0.1-10 g/L or greater, or greater than 6 g/L. In some embodiments of the herein disclosed invention the functional bioactive protein component is Endo-β-N-Acetylglucosaminidase. Such Endo-β-N-Acetylglucosaminidase may be a recombinant protein, homologous to that found in B. infantis. Such recombinant Endo-β—N-Acetylglucosaminidase may further exhibit an amino acid sequence which is at least 80%, at least 85%, at least 90%, at least 95%, or at least 99% identical to the extracellular domain Endo-β—N-Acetylglucosaminidase found in B. infantis.

Some embodiments of the herein disclosed invention comprise a glycoprotein which is contacted with Endo-β-N-acetylglucosaminidase and the composition comprises deglycosylated protein and released N-glycans.

Some embodiments of the herein disclosed invention comprise a protease which may be selected from trypsin and/or chymotrypsin. Such protease may be present in a concentration of greater than 5.6 μg/L. In some embodiments of the herein disclosed invention the protease is present in a concentration of 0.1 g/kg-5 g/kg.

Some embodiments of this invention include the addition of one or more glycans. These glycans may come from natural sources or they may be synthetically produced. In some embodiments of this invention the glycan included is a Mammalian Milk Oligosaccharide (MMO) of degree of polymerization (DP) 2-8. In more preferred embodiments of this invention MMO is a Human Milk Oligosaccharide (HMO). In some embodiments, the glycan is released from a glycoprotein. In some embodiments, the glycan contains at least one residue of fucose or sialic acid. In some embodiments, the glycan contains at least one mannose residue. In other embodiments, the glycan contains at least one N-acetylglucosamine. In some embodiments, the glycan contains galactooligosacharide (GOS) fructoologosaccharide (FOS) or xylooligosacharide (XOS).

The glycans in the composition can include one or more of lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof. The glycans may include oligosaccharides such as: (a) one or more Type II oligosaccharide core where representative species include LnNT; (b) one or more oligosaccharides containing the Type II core and GOS in 1:5 to 5:1 ratio; (c) one or more oligosaccharides containing the Type II core and 2FL in 1:5 to 5:1 ratio; (d) a combination of (a), (b), and/or (c); (e) include one or more Type I oligosaccharide core where representative species include LNT (f) one or more Type I core and GOS in 1:5 to 5:1 ratio; (g) one or more Type I core and 2FL in 1:5 to 5:1 ratio; and/or (h) a combination of any of (a) to (g) that includes both a type I and type II core. Type I or type II may be isomers of each other. Other type II cores include but are not limited to trifucosyllacto-N-hexaose (TFLNH), LnNH, lacto-N-hexaose (LNH), lacto-N-fucopentaose III (LNFPIII), monofucosylated lacto-N-Hexose III (MFLNHIII), Monofucosylmonosialyllacto-N-hexose (MFMSLNH).

Some embodiments of this invention describe a composition comprising a bacterium of the Bifidobacterium species. The Bifidobacterium may be Bifidobacterium adolescentis, Bifidobacterium animalis, Bifidobacterium animalis subsp. animalis, Bifidobacterium animalis subsp. lactis, B. bifidum, Bifidobacterium breve, Bifidobacterium catenulatum, Bifidobacterium longum subsp. longum, Bifidobacterium longum subsp. suis, Bifidobacterium longum subsp. infantis, B. pseudocatanulatum, Bifidobacterium pseudolongum, or a combination thereof. In some embodiments of this invention the Bifidobacterium species is Bifidobacterium longum subsp. longum (“B. longum”), Bifidobacterium longum subsp. infantis (“B. infantis”), or Bifidobacterium breve (“B. breve”). In some embodiments of this invention the Bifidobacterium present is an activated Bifidobacterium culture. (WO 2016/065324 published Apr. 28, 2016 and WO 2019/143871 published Jul. 25, 2019) (incorporated here by reference). In some embodiments of this invention the Bifidobacterium present, activated or otherwise, is B. infantis EVC001 deposited under ATCC Accession No. PTA-125180. In some embodiment of this invention the B. infantis is capable of delivering EndoBI-1.

In any of the foregoing embodiments, the composition may comprise Bifidobacterium in an amount of 0.1 million-500 billion Colony Forming Units (CFU) per gram of composition. The composition may be in an amount of 0.001-100 billion Colony Forming Units CFU, 0.1 million to 100 million, 1 million to 5 billion, or 5-20 billion CFU per gram of composition. The Bifidobacterium may be in an amount of 0.001, 0.01, 0.1, 1, 5, 15, 20, 25, 30, 35, 40, 45, or 50 billion CFU per gram of composition. The Bifidobacterium may be in an amount of 5-20 billion CFU per gram of composition or 5-20 billion CFU per gram of composition or 0.1 million to 100 million CFU per gram of composition. Some embodiments of the herein disclosed invention may include a composition consisting of lysozyme and/or lactoferrin and a Bifidobacterium. In such compositions the functional bioactive protein(s) component and the commensal organism component are described above.

Any of the compositions described herein may take the form of a pharmaceutical composition, dietary supplement, nutritional packet, or food product. In some embodiments of this invention the food product may comprise infant formula, a milk replacer, an enteral nutritional product, and/or a meal replacer for a mammal. In some embodiments of this invention the composition may take the form of a dry powder, such powder may optionally be suspended in oil.

Further embodiments of this invention may include the herein described compositions in an aqueous solution comprising glycans and functional bioactive proteins and may optionally contain one or more bacterium. In some embodiments of this invention the composition suspended in an aqueous solution may be sterile and stored in a single-use container, such container may or may not take the form of a feeding bottle or a bottle to which a feeding nipple or other delivery device can be attached or is attached.

Compositions disclosed herein may utilize many different forms and delivery mechanisms including, but not limited to, capsule, packet, sachet, foodstuff, lozenge, tablet, optionally an effervescent tablet, enema, suppository, dry powder, dry powder suspended in an oil, chewable composition, syrup, or gel. The compositions may be mixed with soy ingredients, such as but not limited to soy lecithin, soy peptides, soy protein. In other embodiments, the compositions may be mixed with minerals such as, but not limited to calcium phosphate. In yet other embodiments, compositions may be mixed with oils such as but not limited to palm olein, soy, coconut and high oleic sunflower oils. In yet other embodiments, the compositions may be mixed with vitamins such as, but not limited to vitamin A palmitate, vitamin D3, vitamin E acetate, and/or vitamin K.

The invention described herein includes a method intended to alleviate or prevent any instance of gut dysbiosis experienced by the user or patient. Gut dysbiosis treated in this way may include, but is not limited to, the reduction of the population of bacterial species that are considered pathogens or potential pathogens, including Klebsiella, Clostridium, Enterobacter, or Escherichia species.

The invention described herein includes a method intended to establish a healthy gut microbiome in the user. This includes, but is not limited to, the establishment or enrichment of a Bifidobacterium culture in the user's gut microbiome. Further methods described herein include the treatment or prevention of autoimmune disorders. Autoimmune disorders treated in this way may include, but are not limited to: celiac disease, inflammatory bowel diseases (Crohn's, ulcerative colitis), irritable bowel syndrome (IBS), multiple sclerosis (MS), Type 1 diabetes mellitus, Psoriasis, atopic dermatitis, asthma, food allergies, necrotizing enterocolitis (NEC), and/or infections such as C. difficile, late on set sepsis, colic, diaper rash.

Other methods described herein prevent or treat metabolic disorders such as obesity, type II diabetes or issues of nutritional insufficiencies or status including weight gain or acquisition of lean vs. fat tissue (body composition). Other methods prevent or treat conditions such as colic or diaper rash.

Further methods described herein include assisting in the recovery of the gut microbiome of the user following chemotherapy, antibiotic treatment, surgery, or similarly disruptive event on gut health. Further methods described herein may relate to improving growth of mammals by administering the compositions herein described through animal feed. In some embodiments of this invention the user or patient is a mammal. Such mammal may include, but is not limited to, a pig, horse, cow, dog, or cat. In some embodiments of this invention the user or patient is a human. In some embodiments of this invention the user or patient is an infant. In some embodiments of this invention the user or patient is a human infant.

In some embodiments of the herein disclosed invention, the compositions are provided to infant mammals to protect the gut from opportunistic pathogen invasion (i.e., to provide colonization resistance).

In some embodiments, the compositions are provided to infant mammals to lower the pH of the gut. In some embodiments, compositions are used to lower the pH of the gut at a time when the subject is in need of mucosal healing.

In some embodiments, the compositions are provided to mammals to reduce the carriage of antibiotic resistant genes and/or levels of endotoxin and/or chronic gut inflammation. In some embodiments, compositions are used to reduce the carriage of antibiotic resistant genes and/or levels of endotoxin and/or chronic gut inflammation at a time when the subject is in need of mucosal healing.

In some embodiments, where the mammal is a human infant, the compositions are used at a time where their adaptive immune system is developing.

In some embodiments, the compositions are provided to mammals of any age who are in need of a treatment to reduce inflammation in the gut. In some embodiments the mammal is a human and the cause of inflammation can be an acute, chronic disease of autoimmune origin or otherwise, such as, but not limited to, necrotizing enterocolitis, diaper rash, colic, late onset sepsis, inflammatory bowel disease, irritable bowel syndrome (IBS), colitis, gut pathogen overgrowth (e.g., C. difficile), hospital acquired infections, asthma, wheeze, allergic responses, Type I Diabetes, Type II diabetes, celiac disease, Crohn's, disease, ulcerative colitis, multiple sclerosis, psoriasis, and atopic dermatitis.

In any embodiment of the herein disclosed invention the compositions may be provided to an infant mammal at a time when their adaptive immune system is developing.

In any embodiment of the herein disclosed invention the mammal may be a human.

### DESCRIPTION OF FIGURES

FIG. 1. PNGase F and EndoBI-1 are active on different locations relative to the N-glycan core, resulting in predictably altered N-glycan profiles.

FIG. 2. Relative area of acidic complex/hybrid, neutral complex/hybrid and high mannose type of glycans, as well as total found in control (grey) and infants fed EVC001 (teal). (P<0.05, *; P<0.01, **; P<0.001, ***).

Filled circles (mannose,), clear circles (galactose), blue squares (HexNAc), red triangles (Fucose) and purple diamonds (NeuAc)

FIG. 3. Extracted compound chromatograms (ECC) for N-glycans released from Lactoferrin (LF) Immunoglobulin G (IgG). Peaks represent LF N-glycans, IgG N-glycans and mutual N-glycans for both glycoproteins, respectively. For the illustration of N-glycan structures filled circles (mannose), clear circles (galactose), blue squares (HexNAc), red triangles (Fucose) and purple diamonds (NeuAc).

FIG. 4. Maximum optical density (OD max) attained by E. coli DH5a in media containing different concentrations of lactoferrin

FIG. 5. Maximum optical density (OD max) attained by E. coli DH5a in media containing different concentrations of lysozyme

FIG. 6. Maximum optical density (OD max) attained by S. thermophilus TH-4 in media containing different concentrations of lysozyme

FIG. 7. Maximum optical density (OD max) attained by E. coli in media containing different combinations of lysozyme and lactoferrin.

FIG. 8. Maximum optical density (OD max) attained by S. thermophilus in media containing different combinations of lysozyme and lactoferrin.

FIG. 9. Maximum optical density (OD max) attained by B. infantis EVC001 in MRS media containing different combinations of lysozyme

FIG. 10. Maximum optical density (OD max) attained by B. infantis EVC001 in media containing 500 μg/mL of lysozyme and 500 μg/mL of lactoferrin compared to no enzyme control

FIG. 11. Flow chart describing the process of generating the supernatant of B. infantis EVC001 grown on lacto-N-tetraose (LNT).

FIG. 12. Growth curves of E. coli with the addition of B. infantis supernatant to the growth media (RPMI+supernatant) compared to control (RPMI+PBS)

FIG. 13. Flow chart describing the process to determine the effects of the supernatant of B. infantis EVC001 grown on lacto-N-tetraose (LNT) on the growth of E. coli as well as the effect on the activity of bioactive enzymes.

FIG. 14. Colony forming units (CFU) of E. coli cells treated the supernatant in which B. infantis had previously grown in the presence of lacto-n-tetraose compared to control.

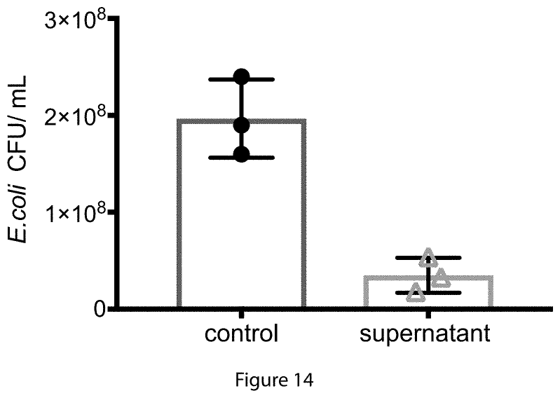

FIG. 15. Growth curves of E. coli treated for 2 hours with the supernatant of B. infantis previously grown on lacto-N-treose compared or control (Phosphate Buffered Saline; PBS)

FIG. 16. Maximum optical density (OD max) of E. coli after treatment for 2 hours with the supernatant of B. infantis previously grown on lacto-N-treose, in media with or without lactoferrin and 500 μg/mL of lysozyme and 500 μg/mL compared to control cells treated for 2 hours with (Phosphate Buffered Saline; PBS)

FIG. 17. Growth rate (h⁻¹) of E. coli after treatment for 2 hours with the supernatant of B. infantis previously grown on lacto-N-treose, in media with or without lactoferrin and 500 μg/mL of lysozyme and 500 μg/mL compared to control cells treated for 2 hours with (Phosphate Buffered Saline; PBS)

### DETAILED DESCRIPTION OF THE INVENTION

Mammalian milk supplies infant mammals with nutritional support for both the infant and the infant's microbiome. Nutritive support for the infant's microbiome is supplied in large part by the glycans called mammalian milk oligosaccharides (MMO). However, healthy development of the microbiome is also facilitated by other components, including bioactive proteins found in mammalian milk or otherwise present in the infant's gastrointestinal tract. The inventors have discovered compositions and methods comprising the use of a functional bioactive protein in conjunction with a glycan and/or a Bifidobacterium strain, which are provided by this invention for addition to the infant's diet or as a therapeutic solution to an individual in need of reducing dysbiosis and/or improving intestinal function.

### Definitions

An “oligosaccharide” is defined as any carbohydrate with 2-20 sugar residues or degrees of polymerization from any source. In some embodiments, it is preferable to have 2-8 sugar residues to include lacto-N-biose.

Mammalian milk oligosaccharide” (MMO) or glycan is defined here as any oligosaccharide that exists naturally in any mammalian milk whether it is its free form or bound to a protein or lipid. MMO and glycans encompass synthetic structures as well as those extracted or purified from sources other than mammalian milk so long as the compound mimics that found in mammalian milk in structure and/or function. That is, while MMOs may be sourced from mammalian milk, they need not be for the purposes of this invention. Sources of MMO may include colostrum products from various animals including, but not limited to cows, goats and other commercial sources of colostrum. It may include MMO enriched from whey permeate, human milk products that are modified through processes such as skimming, protein separation, pasteurization, retort sterilization may also be a source of MMO. MMO includes human milk oligosaccharides.

“Human milk oligosaccharide” (HMO) is defined here as any oligosaccharide which exists in human milk. HMO includes synthetic structures as well as those extracted or purified from sources other than human milk so long as the compound mimics that found in human milk in structure and/or function. That is, while HMOs may be sourced from human milk, they need not be for the purposes of this invention.

A “functional bioactive protein” or “bioactive protein” as used herein is defined as any protein which, unilaterally or in conjunction with other compounds, is capable of effecting functional changes in the infant gut microbiome, these include antimicrobial activity, releasing glycans for use by microorganims (prebiotics), said glycan mixtures and functional bioactive proteins, act as pathogen deflectors or decoy receptors to prevent pathogens from adhering or invading the mucosal surface. Bioactive proteins frequently manifest as enzymes but may include nonenzymatic proteins. Bioactive proteins referenced herein generally are found in mammalian milk but are not limited as such. Digestive enzymes such as proteases, lipases, amylases from any source may be synthesized and/or purified and dried to provide additional function to the individual receiving a composition containing any of the digestive enzymes. Glycoproteins typically found in whey and soy may serve anti-microbial functions, may be a source of prebiotic glycans, and/or may be used to deflect pathogens. Any protein with an effect on the function of the gut microbiome, whether unilaterally or in conjunction with other compounds, is a bioactive protein. While typically these proteins will be mammalian in origin, only the functional effect of the protein is relevant to its classification of a bioactive protein; neither its natural origin nor its source of purification are relevant for the protein's classification as a bioactive protein. Protein is used herein in the same way as is common in the art. A recombinant or synthetic bioactive protein is contemplated whether it is a protein that has been enriched or purified from a natural source or whether the recombinant protein is grown in a micrbial, yeast, algal or other system. The preparation of a new food is synthetic when any of the compositions described herein takes the different components combine them to add function to whatever base food is used to deliver the composition.

“Homologous proteins” referenced herein are defined as any protein, regardless of source or origin, which is the functional and/or structural equivalent of any protein herein referenced, whether currently known or undiscovered. That is, if a protein referenced herein is described as a bioactive protein, any homologous protein currently known or discovered in the future should be assumed to be also referenced and considered a bioactive protein. Any enzyme of this invention may have an amino acid sequence identity that is at least 80%, at least 85%, at least 90%, at least 95%, or at least 99% to the wildtype enzyme.

“Microbiome function” is herein defined as the composite ability of the gut microbiome to utilize available oligosaccharides.

“Infant formula” and “formula” is defined herein as a nutritional composition designed for use by an infant, child, adult or geriatric human.

A “food preparation” is a food that has the compositions described herein formulated to be part of a food. The food preparation includes formulating the compositions during manufacturing and packaging of said food, but may also include preparations that are made just prior to consumption by the individual by adding the composition to an existing food source.

Oil means any edible, food grade oil that is appropriate for the target population

### Bioactive Proteins

MMO's are found in mammalian milk in a milieu of bioactive proteins. The term “bioactive proteins” as used herein is defined as any protein having a biological effect which retains said biological effect while in the digestive system of the subject. Bioactive proteins include lysozyme, proteases, lipases, amylases, lactoferrin, and endoglycosidases. Among these are proteins that have bacteriostatic or bactericidal properties which may play a role in the maintenance of the gut microbiome by diminishing the populations of potentially pathogenic bacteria while not harming favorable ones. Lysozyme, for instance, is a bioactive protein naturally found in milk which has been shown to diminish the presence of potentially pathogenic bacteria in the gut.

Lysozyme is an antimicrobial also known as muramidase, or N-acetylmuramide glycanhydrolase, and is an antimicrobial enzyme produced by animals that forms part of the innate immune system. Lysozyme is a glycoside hydrolase that catalyzes the hydrolysis of 1,4-beta-linkages between N-acetylmuramic acid and N-acetyl-D-glucosamine residues in peptidoglycan, which is the major component of gram-positive bacterial cell wall. Lysozyme is present in human milk, typically at from 0.37-0.89 g/L. The activity of lysozyme in milk, and consequently its bactericidal properties, is significantly reduced by pasteurization.

Lactoferrin is another protein with antimicrobial activity. Lactoferrin is present in human milk, typically at from 2.2-6 g/L. Lactoferricin is a fragment that may be released from Lactoferrin.

Endo-beta-N-Acetylglucosaminidase is a glycosylhydrolase that can cleave N-glycans particularly high mannose, hybrid and/or complex glycans. Other endoglycosidases from any source may be used to cleave O-linked glycans.

Of particular interest, is an Endo-beta-N-Acetylglucosaminidase from B. infantis (EndoBI) that can cleave N-glycans from glycoproteins. Mannosyl-glycoprotein endo-β-N-acetylglucosaminidases or simply endo-β-N-acetylglucosaminidases (ENGase, EC 3.2.1.96) are glycoside hydrolyses that cleave the N,N′-diacetylchitobiosyl unit in high mannose glycopeptides and glycoproteins containing the -[Man(GlcNAc)₂]Asn-structure. In this invention endo-β-N-acetylglucosaminidases are considered those that are found in B. infantis and recombinant versions of those. They may be referred to as Endo BI, EndoBI-1 and Endo BI-2. EndoBI-1 enzyme includes a signal helix (1-36), the active ENGase (37-517), and a transmembrane helix (518-545). Similarly, EndoBI-2 enzyme includes a signal peptide (1-60), the active ENGase (61-515), and a gram positive LPXTG cell wall anchor helix (520-555). The masses of the complete enzymes were calculated as 56.1 and 59.6 kDa respectively with Compute pI/Mw tool of SIB ExPASy Bioinformatics Resource Portal. The ENGase part of the enzymes consist two discrete domains similar to other GH18 members. First there is an N-terminal glycosidase domain for both enzymes. The glycosidase domain of EndoBI-1 spans through amino acid residues 51-366 whereas EndoBI-2 61-360. The second domain of the enzymes is a substrate binding domain which contains a potential carbohydrate binding module (CBM) and a 4-helix up-down bundle domain which is similar to other members of the GH18 family.

A. EndoBI-1 Fasta sequence - full length protein
>ACJ53522.1 glycosyl hydrolase, family 20
[Bifidobacterium longum subsp. infantis ATCC
15697 = JCM 1222 = DSM 20088]
MTFIKQMMPRYVASMTAGIVAAAMAATCAFAPVANADAVSPTQETIQSTG
RHFMVYYRAWRDVTMKGVNTDLPDDNWISMYDIPYGVDVVNIFSYVPSGQ
EEQAQPFYDKLKSDYAPYLHSRGIKLVRGIDYTGVAVNGFRTFMKEQNKT
ESEATEADYDAYAKQVIDKYMISVGLDGLDIDMEAHPNDADVKISDNVIR
ALSKHIGPKSAKPDTTMFLYDTNGSYLNPFKNVAECFDYVAYQQYGSSSD
RTARAAADYQPYIGNEFVPGLTFPEEGDMNNRWYDATEPYEESHFYQVAS
YVREHNLGGMFVYALDRDGRNYDEDLRRIVPSNLLWTKTAIAESEGMALD
TAKTAANHYLDRMSLRQVIDDNAASADKARDMVGKAANLYETNKAVLGGD
YGEGFSNTYDPTLEAGLLGIDISVLQQQIDKSSEIIGADTAESDAKTALR
MARDAAIDGLTGKIYTADQVSAWSQALKAALDATVPVPTPDSTDQNGNRD
KVTNHKVQGQPKQLSATGISTDIIVAVGVTLAIAGVALSLSRKLS
B. EndoBI-2 Fasta sequence - full length protein
>BAJ71450.1 glycosyl hydrolase [Bifidobacterium
longum subsp. infantis 157F]
MCCSQGSEGRSSIGRRLAAVAAGVVATALMLGCSLAMGTVANAQEGESPV
AASQEGNGNKHFMVYYRAWRDVTMKGVNTDLPDDNWISMYDIPYGIDVVN
VFSYVPSGQEAAAQPFYDKLKSDYAPYLHARGIKLVRGLDYSGVMVDGFK
TWIAQQGKNVDSATESDYDAYADHVIETYMTSVGLDGLDIDMETFPDAAQ
VAISDQVITALAKRIGPKSDNPEGTMFLYDTNGSYTAPFKNVSDCFDYVA
YQQYGSDSNRTAKAAATYEQFIDSTKFVPGLTFPEEGDMNNRWNDATEPY
LDSHFYDVASYSYDHNLGGMFVYALDRDGRTYSDDDLAHIKPSNLIWTKT
AIAQSQGMSLENAKQAANHFLDRMSYTKDVPAETRQTVAAATNLYEVDKA
VLGADWNDGYSNTYDPTLELSLTSIDTTALTGAIAKADALLADGATDTDV
RTTLTTARNAAVTGLTSKLYTGADVVSWTASLNTAIADATGGKPKPQQPG
TGETDKPSSGDASHNKPQSATGRLASTGSDGTVVLSVAVIMTLAGISVFA
VRRRG

In particular, B. infantis has specific endo-β-N-acetylglucosaminidases (EndoBI) that are of interest as bioactive proteins. These proteins are designated EndoBI-1 and EndoBI-2. The extracellular domain of Endo BI-1 is defined as “NADAVSPTQETIQSTGRHFMVYYRAWRDVTMKGVNTDLPDDNWISMYDIPYGVD VVNIFSYVPSGQEEQAQPFYDKLKSDYAPYLHSRGIKLVRGIDYTGVAVNGFRTFMK EQNKTESEATEADYDAYAKQVIDKYMISVGLDGLDIDMEAHPNDADVKISDNVIRA LSKHIGPKSAKPDTTMFLYDTNGSYLNPFKNVAECFDYVAYQQYGSSSDRTARAAA DYQPYIGNEFVPGLTFPEEGDMNNRWYDATEPYEESHFYQVASYVREHNLGGMFV YALDRDGRNYDEDLRRIVPSNLLWTKTAIAESEGMALDTAKTAANHYLDRMSLRQ VIDDNAASADKARDMVGKAANLYETNKAVLGGDYGEGFSNTYDPTLEAGLLGIDIS VLQQQIDKSSEIIGADTAESDAKTALRMARDAAIDGLTGKIYTADQVSAWSQALKAA LDATVPVPTPDSTDQNGNRDKVTNHKVQGQPKQLSAT” for the purposes of establishing homology. The endoBI2 sequence defined for the purposes of homology is VANAQEGDSPVAASQEGNGNKHFMVYYRAWRDVTMKGVNTDLPDDNWISMYDIP YGIDVVNVFSYVPSGQEAAAQPFYDKLKSDYAPYLHARGVKLVRGLDYSGVMVDG FKTWIAQQGKNVDSATESDYDAYADHVIETYMTSVGLDGLDIDMETFPDAAQVAIS DQVITALAKRIGPKSDNPEGTMFLYDTNGSYTAPFKNVSDCFDYVAYQQYGSDSNR TAKAAATYEQFIDSTKFVPGLTFPEEGDMNNRWNDATEPYLDSHFYDVASYSYDHN LGGMFVYALDRDGRTYSDDDLAHIKPSNLIWTKTAIAQSQGMSLENAKQAANHFLD RMSYTKDVPAETRQTVAAATNLYEVNKAVLGADWNDGYSNTYDPTLELSLASIDTT ALTGAIAKADALLADGATDTDVRTTLTTARNAA. Homologous proteins for Endo BI-1 or EndoBI-2 of this invention may have an amino acid sequence identity that is at least 80%, at least 85%, at least 90%, at least 95%, or at least 99% identical.

In some embodiments, an EndoBI has modified active site to have a pH optimum of more than 4, more than 5, more than 6, more than 7, more than 8. In some embodiments, change in pH optimum is achieved by replacing the glutamic acid in the active site. The replacement may be selected from glutamine (Gln, Q), Aspartic acid (Asp, D), Serine (Ser, S), Lysine (Lys, K), Arginine (Arg, R), Histidine (His, H), Threonine (Thr, T), Tyrosine (Tyr, Y) or Cystenine (Cys, C).

### Glycans

Glycans for use in the compositions of this invention are typically MMO (oligosaccharides found in any mammalian milk including, but not limited to human, bovine, goat) and may be free oligosaccharides, or glycans bound to protein or lipid, or the same glycans released from the protein or lipid. Oligosaccharides of use in the present invention can include one or more of lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof. The oligosaccharides may include: (a) include one or more Type II oligosaccharide core where representative species include LnNT; (b) one or more oligosaccharides containing the Type II core and PDX, maltodextrin, inulin GOS, FOS, or XOS in 1:5 to 5:1 ratio; (c) one or more oligosaccharides containing the Type II core and 2FL in 1:5 to 5:1 ratio; (d) a combination of (a), (b), and/or (c); (e) include one or more Type I oligosaccharide core where representative species include LNT (f) one or more Type I core and GOS, FOS, or XOS in 1:5 to 5:1 ratio; (g) one or more Type I core and 2FL in 1:5 to 5:1 ratio; and/or (h) a combination of any of (a) to (g) that includes both a type I and type II core. Type I or type II may be isomers of each other. Other type II cores include but are not limited to trifucosyllacto-N-hexaose (TFLNH), LnNH, lacto-N-hexaose (LNH), lacto-N-fucopentaose III (LNFPIII), monofucosylated lacto-N-Hexose III (MFLNHIII), Monofucosylmonosialyllacto-N-hexose (MFMSLNH).

In other embodiments, a prebiotic or other excipient such as, but not limited to galactooligosaccharide (GOS), fructooligosaccharide (FOS), xylooligosaccharide (XOS), polydextrose (PDX), Raffinose, and maltodextrin may be used in place of or together with any mammalian milk oligosaccharides.

In some embodiments, the DP4 is at least 30% of the total GOS provided. In others D4 and D5 make up at least 50% of the GOS Composition. In some embodiments, the GOS has less than 10% DP3 (WO 2010/105207, published Sep. 16, 2010 incorporated here by reference). In some embodiments, a ratio of GOS/FOS, GOS/inulin, GOS/FOS/inulin, GOS/PDX is used with one or more mammalian milk oligosaccharides. In some embodiments, the GOS/FOS may be limited to DP2-3

In some embodiments, glycans are released from glycoproteins by chemical or enzymatic means. Glycoproteins may be from any mammalian milk such as, but not limited to, human or bovine milk. The glycoproteins may be from plants including soymeal.

### Compositions

In some embodiments, a B. infantis is combined with MMO to produce acetate and lactate (WO 2018/006080 filed Jan. 4, 2018 incorporated here in by reference) and may further comprise lysozyme and/or lactoferrin.

In some embodiments, a composition of MMO with a recombinant lysozyme and/or lactoferrin further comprises B. infantis. The use of such composition enhances the growth and colonization of B. infantis, such that the relative abundance of Bifidobacterium increases to at least 65%, at least 75%, at least 85, or at least 90% of the total microbiome; whereas as an example enterobacteriacaeae decreases to less than 20%, less than 15%, less than 10%, or less than 5% of the total microbiome.

In some embodiments, a composition comprises a Lactobacillus reuteri with raffinose, and further comprises a recombinant lysozyme and/or lactoferrin. In some embodiments, a composition comprises a Lactobacillus rhamnosus (LGG) and DP2-3 GOS or FOS and may further comprise Lysozume and.or lactoferrin.

Nutritive compositions according to this invention typically contain at least a plurality of glycans and one or more bioactive proteins. Typically, such compositions contain other nutritive components, such as sugars, lipids, vitamins, minerals, and the compositions may also include other bioactive components. Nutritive compositions may further comprise the compositions of (Nutritive compositions with secretory IgA, milk fat globule membrane components and/or Bifidobacterium; U.S. provisional application filing on Jul. 26, 2019) and probiotic strains described in (WO 2019/232284, published Dec. 5, 2019 and incorporated herein by reference). These compositions may be in solid form, such as a powder, or in liquid form, such as an aqueous suspension. End use of the composition is generally in liquid form for administration to subjects in need.

Oils may be selected from any food-grade oil from any source whether natural originating in a plant, animal, or microbe; or synthetically created. In preferred embodiments of this invention the oil is selected from medium chain triglyceride (MCT) oil, sunflower oil, docosahexaenoic acid (DHA) or arachidonic acid (ARA)-containing oils, and/or mineral oil.

In some embodiment the compositions are provided to mammals of any age who are in need of a treatment to reduce inflammation in the gut or otherwise improve gut health.

The compositions may be tailored or targeted to specific age groups, such as a preterm infant who may be born with a gestational age of less than 33 weeks, the preterm babies may be a very low birth weight (VLBW), or low birth weight (LBW), a term infant (0-3 months), an infant 3-6 months, an infant (6-12 months), a weaning infant (4-12 months), a weaned infant (12 months to 2 years) and child (1-16 years), an adult (16-70 yr), or an older adult (70-100+yr).

Any compositions described herein are provided daily for at least 1 day, at least 3, at least 7, at least 14, at least 28 days, at least 3 months, at least 6 months or at least 12 months to any subject in need of. In some embodiments, infants are fed MFGM complex compositions when the adaptive immune system is developing preferably starting at birth, in the first 100 days of life, the first 6 months of life or in the first year of life wherein the compositions are provided daily for at least 1 day, at least 3, at least 7, at least 14, at least 28, at least 3 months, at least 6 months or at least 12 months.

In some embodiments, the compositions are provided to subjects to protect the gut from opportunistic pathogen invasion or for recovery after such invasion.

In some embodiments, the compositions are provided to infant mammals to lower the pH of the gut at a time where their adaptive immune system is developing. In a preferred embodiment the infant is a human infant from age 0-24 months. In some embodiments, compositions are used to lower the pH of the gut at a time when the subjects is in need of mucosal healing.

In another embodiment, the compositions are provided to infant mammals to reduce the carriage of antibiotic resistant genes and/or levels of endotoxin and/or chronic gut inflammation at a time where their adaptive immune system is developing. In a preferred embodiment the infant is a human infant from age 0-24 months. In some embodiments, compositions are used to reduce the carriage of antibiotic resistant genes and/or levels of endotoxin and/or chronic gut inflammation at a time when the subject is in need of mucosal healing.

Administration of such methods and compositions may improve the growth rate of the mammal measured by weight gain (kilograms/day), Z scores, such as weight for age (WAZ), length for age (LAZ) or (weight for length) WLZ.

In another embodiment, the compositions are provided to mammals of any age who are in need of a treatment to reduce inflammation in the gut. In a preferred embodiment the mammal is a human and the cause of inflammation can be an acute, chronic disease of autoimmune origin or otherwise, such as, but not limited to, necrotizing enterocolitis, diaper rash, colic, late onset sepsis, inflammatory bowel disease, irritable bowel syndrome (IBS), colitis, gut pathogen overgrowth (e.g., C. difficile), hospital acquired infections, asthma, wheeze, allergic responses, Type I Diabetes, Type II diabetes, celiac disease, crohn's, disease, ulcerative colitis, multiple sclerosis, psoriasis, and atopic dermatitis.

In another embodiment the compositions can be provided to a non-human mammal of any age including, but not limited to pigs, cows, horses, dogs, cats, donkeys, camels, sheep, goats and rabbits. In another embodiment, the compositions are provided to non-human mammals for the prevention or treatment of gut inflammatory conditions. The non-human mammals may be newborn mammals, who are optionally nursing, or they may be food production animals, performance animals or domestic animals.

### EXAMPLES

### Example 1. Releasing N-Glycans from Soymeal Proteins Using EndoBI-1

Preparation of soy-meal proteins for enzymatic digestion: To remove free monosaccharides/oligosaccharides and other contaminants that might hinder enzyme activity, proteins were precipitated using cold ethanol (4:1 v/v ratio). Initially, 20 ml of soy-meal protein mixture (10 mg/ml in DI water) was mixed with 80 mL of cold ethanol and incubated for 1 h at −20° C. Then the mixture was centrifuged for 15 min at max speed. The supernatant containing free monosaccharides/oligosaccharides and other contaminants was discarded and the pellet was resuspended in 20 mL of DI water and the protein solution was stored at −20° C. for further analysis. The cleanup process was repeated three times to remove all unwanted contaminants. If necessary, the purity of proteins can be tested by MALDI-TOF Mass Spectrometry.

Digestion of soymeal proteins by EndoBI-1 and visualization by SDS-PAGE gel mobility assay: Purified soymeal proteins were resuspended in water to a concentration of 5 mg/ml and incubated for 5 min at 95° C. to denature the proteins. To a 1.0 ml aliquot of the denatured protein, 1.0 ml of sodium phosphate buffer at pH 5.0 and 90 uL of an EndoB-1 solution (5 mg/ml) were added, and the mixture incubated at 37° C. for 1 h to allow deglycosylation of the soy proteins. Aliquots of this soymeal protein/enzyme solution were then subjected to SDS-PAGE on 5%42% gradient gel and the extend of the deglycosylation was visualized.

pH and temperature optimization of glycan release from soy meal proteins by EndoBI-1: To optimize the reaction conditions of EndoBI-1 on soy meal proteins, various pH and temperature values were combined and released glycans were measured by phenol sulphuric total carbohydrate assay where the mannose was used for the standard curve. Based on the findings, the activity of EndoBI-1 on soy meal proteins is mostly dependent on temperature. It was shown that the enzyme's activity was not affected by pH, whereas activity is positively correlated with increased temperature (Table 1). The results suggest that the enzyme can maintain its high activity on a wider variety of pH values on soy meal proteins.

TABLE 1
Glycan release efficacy on different pH and
temperature values

| | | Incubation | Glycan |
|---|---|---|---|
| Treatment | | temperature | Yield |
| Code | pH | (° C.) | (ug) |
| 1 | 4.5 | 24 | 22 +/− 3 |
| 2 | 4.5 | 30 | 60 +/− 7 |
| 3 | 4.5 | 37 | 72 +/− 6 |
| 4 | 4.5 | 42 | 75 +/− 7 |
| 5 | 5 | 24 | 24 +/− 4 |
| 6 | 5 | 30 | 63 +/− 7 |
| 7 | 5 | 37 | 74 +/− 12 |
| 8 | 5 | 42 | 74 +/− 10 |
| 9 | 5.5 | 24 | 26 +/− 6 |
| 10 | 5.5 | 30 | 66 +/− 12 |
| 11 | 5.5 | 37 | 70 +/− 11 |
| 12 | 5.5 | 42 | 72 +/− 8 |
| 13 | 6 | 24 | 25 +/− 5 |
| 14 | 6 | 30 | 58 +/− 7 |
| 15 | 6 | 37 | 65 +/− 8 |
| 16 | 6 | 42 | 70 /− 9 |

Utilization of released N-glycans from soy glycoproteins by B. infantis. To test the consumption of N-glycans released from glycoproteins, RMPI 1640 medium (without glucose) with 2% (w/v) freed glycans was used for the B. infantis growth assay. As a negative control RMPI 1640 without a carbohydrate source and a positive control RPMI 1640 with 2% (w/v) glucose were used. Based on the results, B. infantis showed growth (final OD=0.45) on RMPI 1640 supplemented with 2% glycans (Table 2).

TABLE 2
B. infantis growth on released glycans from soymeal proteins

| | No Carbon Source | Glucose | Glycans |
|---|---|---|---|
| Max OD | — | 1.4 | 0.45 |

The experiment described in example 1 is not limited to soymeal protein. One skilled in the art will recognize that the same experimental design can be used for any plant, animal, fungal, or insect glycoprotein.

### Example 2. EndoBI-1 in B. infantis Rescues Glycan Energy in the Colon of a Breast-Fed Baby

A clinical trial was established with newborn breast-fed babies who were supplemented with daily doses for 21 days of Bifidobacterium longum subsp. infantis (EVC001 or Evivo, supplied by Evolve BioSystems Corp, Davis Calif., USA at a dose of 1.8×10¹⁰ CFU/day) vs. a control group without supplementation. Fecal samples were collected from the two groups of infants on day 28 of life and profiled by untargeted mass spectrometry. Neither the subjects, nor their mothers differed significantly (P>0.05) across the demographics including delivery mode (cesarean section or vaginally), duration of labor, antibiotic administration to mothers during labor, complications associated with labor, gestational age at delivery, birth weight or length, sex, provision of antibiotics to infants, pre-pregnancy BMI, weight gain during pregnancy, group-B Streptococcus diagnosis, or maternal age [Karav, 2019 Journal of Functional Foods, Volume 61, October 2019, 103485].

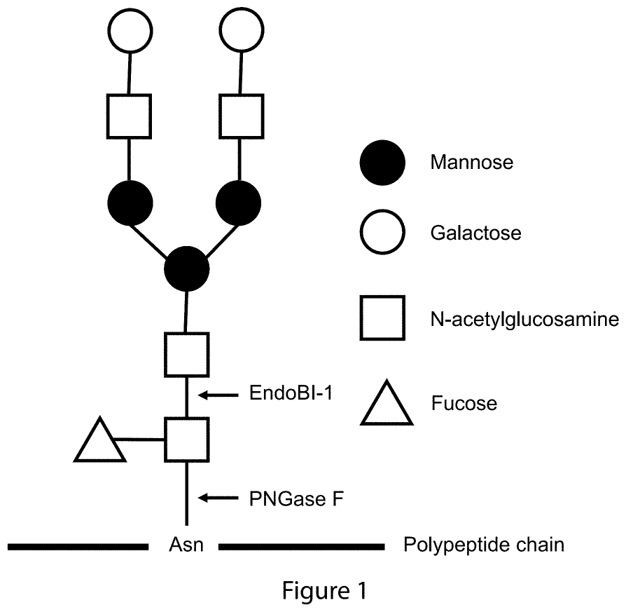

Untargeted mass spectra of glycans from fecal samples were collected and analyzed using Agilent Mass Hunter Workstation Data Acquisition Version B.02.01 on the nanoHPLC-chip/TOF. The “Find Compounds by Molecular Feature” function of the software was used to identify N-glycan structures released from human milk glycoproteins by B. infantis EndoBI-1 (endo-β-N-acetlyglucosaminidase). The software generated extracted compound chromatograms in the range of 400 to 3,000 m/z, with an ion count cutoff of 600, allowed charge states of 1 to 3, retention times of 5 to 40 min, and a typical isotopic distribution of small biological molecules. To determine target N-glycan compositions, previously published libraries were used. PNGase F cleaves the bond between the primary N-acetylhexosamine (HexNAc) and the polypeptide, whereas EndoBI-1 cleaves between the HexNAc bound to the polypeptide and the second HexNAc (FIG. 1). PNGase F activity is limited to when a fucose is attached to the primary HexNAc. However, EndoBI-1 is not affected by core N-glycan fucosylation, which results in the release of a broader diversity of N-glycan structures. Compound abundances were expressed as volume in ion counts that corresponded to absolute abundances of the compounds in each sample.

To assess how variations in bacterial communities in the infant gut were related to N-glycan profiles, sequencing of 16S rDNA amplicons was performed on an Illumina MiSeq sequencer. Differences in bacterial community composition and N-glycans were calculated using principle coordinate analysis (PCoA) and a Bray-Curtis dissimilarity index between all N-glycan species was visualized via PCoA. To evaluate the effect-size of EVC001 colonization, both weighted UniFrac and Bray-Curtis dissimilarity matrices were tested via Permanova multivariate comparisons with 999 permutations and FDR-corrected P-values. N-glycan abundance was transformed to dissimilarity matrices using Euclidean distance while phylogenetic distance was obtained via the weighted UniFrac algorithm. Tests were performed using Pearson's product-moment correlation coefficient (r) with 999 permutations and a two-tailed test.

N-glycan compositions were determined by the untargeted approach of nano-HPLC-Chip-TOF. And thirty structures (representing 49 total isomers) were detected. Infants fed EVC001 had a significantly higher number of distinct N-glycan structures (42.37+/−7.24) than the control infants not colonized by EVC001 (3.8+/−2.82) (Table 4). Among EVC001-colonized infants, an average of 25+/−3.13 SD of them were neutral complex/hybrid, 13.5+/−4.24 SD were sialylated complex/hybrid and 3.25+/−2.71 SD were high mannose type of N-glycans. In contrast, control infants had a fecal N-glycome composed of neutral complex/hybrid N-glycans (3.1+/−2.06), with only 0.1 (+/−0.33) acidic complex/hybrid N-glycans and 0.7 (+/−0.97) high mannose structures.

TABLE 4
N-glycan families detected in fecal samples. The mean (+/−SD)
number of N-glycan isomers found in fecal samples of infants
fed EVC001 and control subjects.

| Number of N-glycans | Control | EVC001-fed | FDR-adjusted |
|---|---|---|---|
| detected | (+/−SD) | (+/−SD) | P-value |
| Total N-glycans | 3.8 +/− 2.82 | 42.37 +/− 7.24 | P < 0.001 |
| Neutral Complex/Hybrid | 3.1 +/− 2.38 | 25.125 +/− 3.14 | P < 0.001 |
| Acidic (Sialylated) | 0.1 +/− 0.32 | 13.5 +/− 4.24 | P < 0.001 |
| Complex/Hybrid | | | |
| High Mannose | 0.7 +/− 0.95 | 3.25 +/−2.71 | P < 0.05 |

The relative abundance of each class of N-glycan compounds was determined for EVC001-fed infants and control infants (Table 5). Based on these findings, the relative abundance of samples from infants colonized with EVC001 and control samples were ranged between 163273+/−204568 to 27509632+/−24168794 and 0 to 1986832+/−1994415, respectively. The most highly abundant compounds detected in EVC001 were 43000, 43100, 53000, 53100, 53200, 53010 and 53310. These compounds are different than a previously performed analysis based on PNGAse F by Dallas et al. (Dallas et al., 2011) which showed the highest abundant N-glycan compositions are 5Hex-2Fuc-4HexNAc-1NeuAc, 5Hex-1Fuc-4HexNAc-1NeuAc, 5Hex-2Fuc-4HexNAc and 5Hex-3Fuc-4HexNAc. Differences observed were accounted for by a single HexNAc given a difference in enzyme activity and substrate specificity between PNGase F, used by Dallas et al (Dallas et al., 2011), and EndoBI-1.

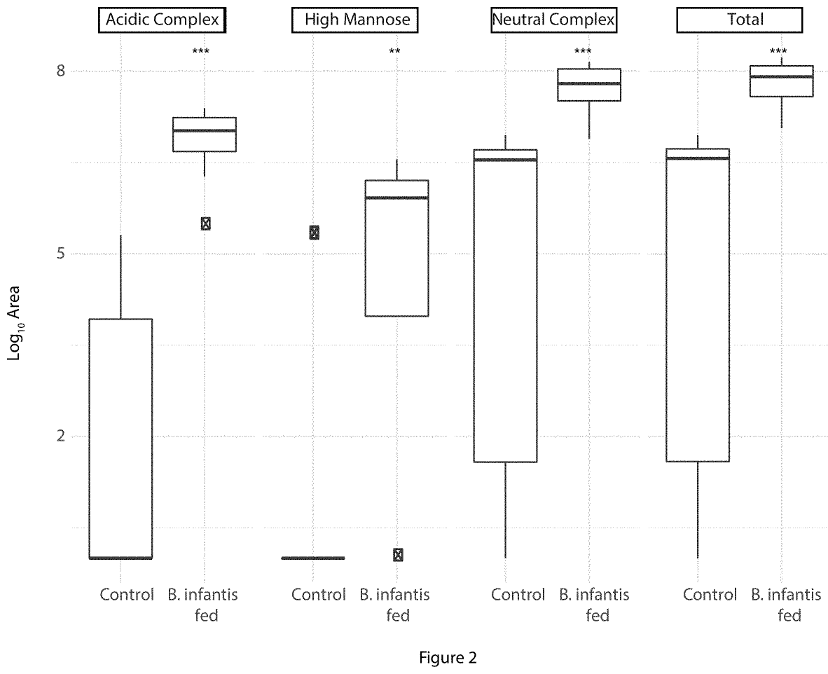

The relative abundance of each class of N-glycans was determined for EVC001-fed infants and control samples are shown in Table 5. Based on the results, the total relative abundance of neutral complex/hybrid, sialylated complex/hybrid and high mannose glycans of EVC001 fed samples were 69,478,405, 11,533,305 and 1,232,719, respectively, whereas these values were 3,511,737, 56,810 and 39,322 for control samples (FIG. 2).

TABLE 5
Abundance (peak volume) of N-glycans found in EVC001 fed and control
samples. Compositions are ordered by Hex-HexNAc-Fuc-NeuAc-NeuGc

| | | | | | | | | FDR- |
|---|---|---|---|---|---|---|---|---|
| | N-glycan | | | | | Volume | Volume | adjusted |
| N-glycan type | Code | Composition | m/z | z | m | Control | EVC001 Fed | P Value |
| Neutral | 1 | 41000 | 436.74 | 2 | 871.48 | 0 +/− 0 | 984402 +/− 539230 | 0.0037 |
| Complex/Hybrid | 2 | 52000 | 618.23 | 2 | 1236.45 | 0 +/− 0 | 727585 +/− 740910 | 0.0196 |
| | 3 | 43000 | 639.74 | 2 | 1277.47 | 0 +/− 0 | 16336429 +/− 12518995 | 0.0037 |
| | 4 | 43100 | 712.76 | 2 | 1423.53 | 342923 +/− 731341 | 11473565 +/− 7892814 | 0.0037 |
| | 5 | 53000 | 720.77 | 2 | 1439.53 | 256653 +/− 541647 | 27509632 +/− 24168794 | 0.0088 |
| | 6 | 52200 | 764.33 | 2 | 1526.66 | 0 +/− 0 | 170903 +/− 267358 | 0.0568 |
| | 7 | 53100 | 793.80 | 2 | 1585.59 | 925328 +/− 1334063 | 4347701 +/− 5648944 | 0.0986 |
| | 8 | 53200 | 866.83 | 2 | 1731.64 | 1986832 +/− 1994415 | 5545213 +/− 5803780 | 0.1290 |
| | 9 | 63100 | 874.33 | 2 | 1747.68 | 0 +/− 0 | 311166 +/− 442663 | 0.0568 |
| | 10 | 35200 | 907.12 | 2 | 1825 | 0 +/− 0 | 306844 +/− 402042 | 0.0260 |
| | 11 | 45100 | 915.18 | 2 | 1829.49 | 0 +/− 0 | 209082 +/− 277573 | 0.0260 |
| | 12 | 45200 | 988.24 | 2 | 1975.4 | 0 +/− 0 | 256041 +/− 346230 | 0.0260 |
| | 13 | 36200 | 1008.81 | 2 | 2015.62 | 0 +/− 0 | 687310 +/− 832357 | 0.0260 |
| | 14 | 65100 | 1077.90 | 2 | 2153.79 | 0 +/− 0 | 458324 +/− 711077 | 0.0260 |
| | 15 | 36300 | 1081.77 | 2 | 2161.55 | 0 +/− 0 | 154200 +/− 173652 | 0.0260 |
| Acidic | 16 | 52010 | 764.33 | 2 | 1526.66 | 0 +/− 0 | 568612 +/− 870952 | 0.0196 |
| (Sialylated) | 17 | 64310 | 811.11 | 3 | 2534.2 | 0 +/− 0 | 942611 +/− 1506753 | 0.0568 |
| | 18 | 52110 | 837.83 | 2 | 1673.65 | 0 +/− 0 | 163273 +/− 204568 | 0.0260 |
| | 19 | 62010 | 845.65 | 2 | 1689.29 | 0 +/− 0 | 406477 +/− 797280 | 0.0260 |
| | 20 | 44010 | 886.82 | 2 | 1771.64 | 0 +/− 0 | 613252 +/− 1069722 | 0.0568 |
| | 21 | 53010 | 866.31 | 2 | 1731.62 | 17234 +/− 54499 | 2263998 +/− 3337414 | 0.0196 |
| | 22 | 53110 | 939.34 | 2 | 1876.67 | 0 +/− 0 | 788923 +/− 1468401 | 0.1309 |
| Complex/Hybrid | 23 | 44110 | 959.85 | 2 | 1917.70 | 0 +/− 0 | 817721 +/− 1201857 | 0.0196 |
| | 24 | 53210 | 1012.37 | 2 | 2023.73 | 20230.4 +/− 63974 | 2374434 +/−3643257 | 0.0260 |
| | 25 | 53310 | 1085.90 | 2 | 2169.79 | 19345 +/− 61175 | 2594002 +/− 3763059 | 0.0260 |
| High | 26 | 51000 | 1034.37 | 1 | 1033.37 | 19213 +/− 60758 | 191175 +/− 294378 | 0.1481 |
| Mannose | 27 | 61000 | 598.21 | 2 | 1195.42 | 0 +/− 0 | 400248 +/− 573039 | 0.0568 |
| | 28 | 81000 | 760.09 | 2 | 1519.1 | 20108 +/− 63589 | 207358 +/− 330429 | 0.1796 |
| | 29 | 91000 | 841.13 | 2 | 1681.25 | 0 +/− 0 | 433937 +/− 481589 | 0.0260 |
| Neutral Complex | | | | | | 3511738 +/− 3085141 | 69478405 +/− 48954788 | <0.001 |
| Acidic Complex | | | | | | 56810 +/− 91761 | 11533306 +/− 8831971 | <0.001 |
| High Mannose | | | | | | 39322 +/− 82925 | 1232719 +/− 1377197 | <0.01 |
| All Glycans | | | | | | 3607870 +/− 3096536 | 82244430 +/− 55275913 | <0.001 |

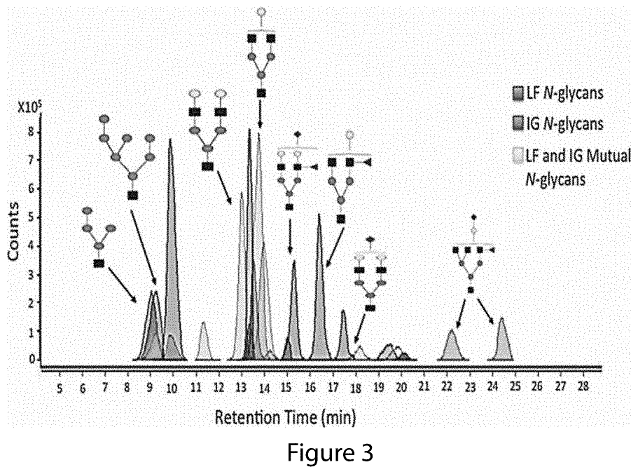

When the compounds detected in our study were compared with the lactoferrin N-glycan library, 14 out 18 N-glycans found in stool from EVC001-fed infants could be attributed to lactoferrin glycans while a minority of these glycans were of nonspecific origin (i.e. they could originate from any one of multiple N-linked glycoproteins) or originated from milk immunoglobulins. These findings show that the majority of glycans detected in EVC001 fed samples originated from glycosylated lactoferrin and glycosylated immunoglobulins (FIG. 3) indicating an unexpected enrichment of glycans that can be consumed by B. infantis. These results establish that B. infantis releases a substantial fraction of the N-glycans found on glycosylated proteins in the colon as a method of rescuing and metabolizing any remaining energy prior to its exit from the body.

### Example 3. In Vitro Screening for Susceptibility of Dysbiotic Strains to Lysozyme and Lactoferrin

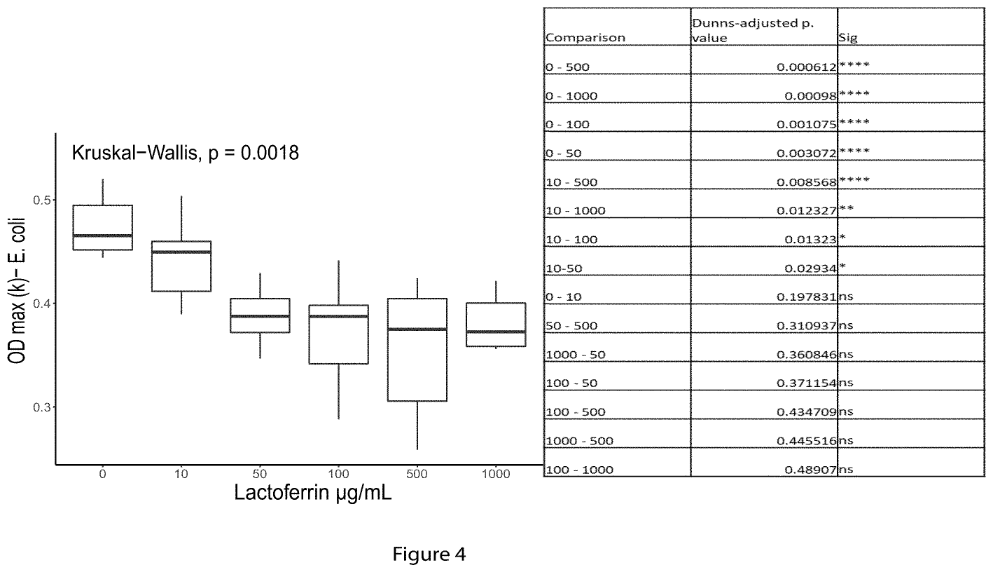

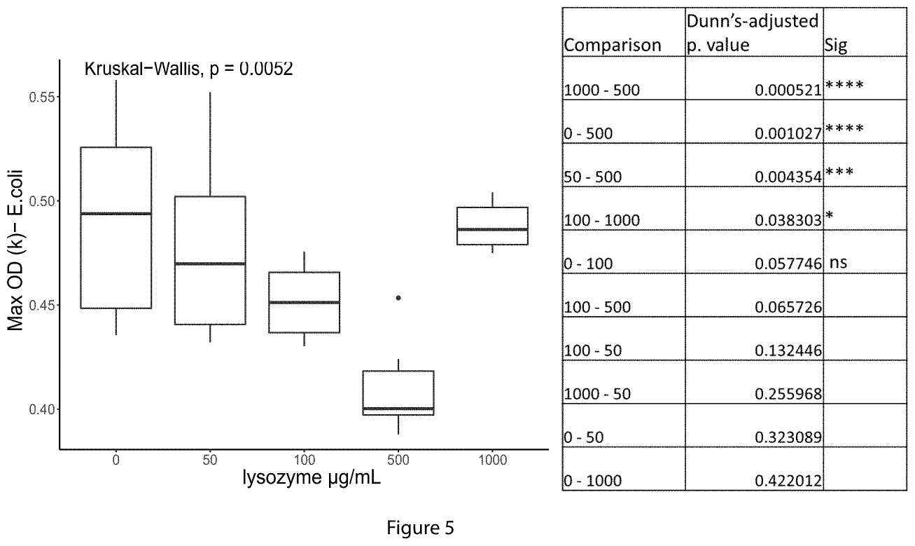

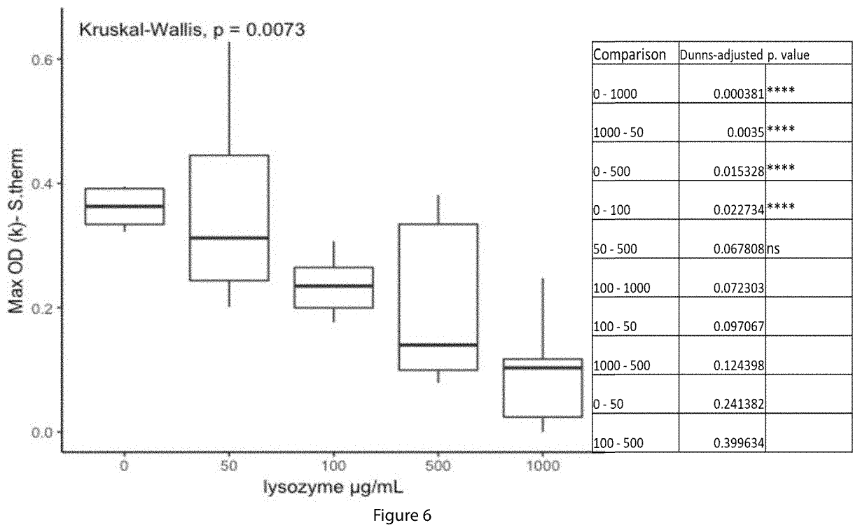

The tolerance of representative strains of taxa associated with dysbiosis (Table 3) was determined by inoculating growth medium containing various concentrations of lysozyme or lactoferrin using the microdilution method. Briefly, overnight cultures were diluted 1:100 in fresh growth media. The bacterial solution was mixed with a stock solution of lactoferrin (10, 50, 100, 500 and 1000 μg/ml) or a stock solution of lysozyme (10, 50, 100, 500 and 1000 μg/ml or Phosphate Buffered-Saline PBS (control) in wells of a 96-well microplate. Microplates were incubated at 37° C. Bacterial growth was monitored every 30 minutes by optical density OD₆₀₀ over a time-course of twenty four hours. Data was generated for six biological replicates. Microbial growth kinetics were analyzed by plotting the optical density (OD₆₀₀) against time. The data was fitted to the logistic equation of bacterial growth, and highest bacterial growth density attained (OD max) was compared between enzyme concentrations are compared to an untreated control. The ODmax of attained by E. coli was significantly lower (P<0.05; Kruskal-Wallis-Dunn's-adjusted) when 50, 100, 500 and 1000 μg/ml of lactoferrin were added to the growth media compared to the control (FIG. 4). The OD max of E. coli was also significantly reduced when 500 μg/ml of lysozyme was added the media compared to control. Addition of 50 and 100 μg/ml of lysozyme to the resulted in reduced ODmax for E. coli a but did not reach statistical significance(P<0.05; Kruskal-Wallis-Dunn's-adjusted) (FIG. 5). Similarly, the OD max of attained by S. thermophilus, was significantly lower (P<0.05; Kruskal-Wallis-Dunn's-adjusted) under the presence of 10, 100, 500 and 1000 μg/mL of lysozyme in the growth media compared to control (no enzyme) (FIG. 6). These results suggest lactoferrin and lysozyme can significantly reduce the growth of representative taxa associated with dysbiosis.

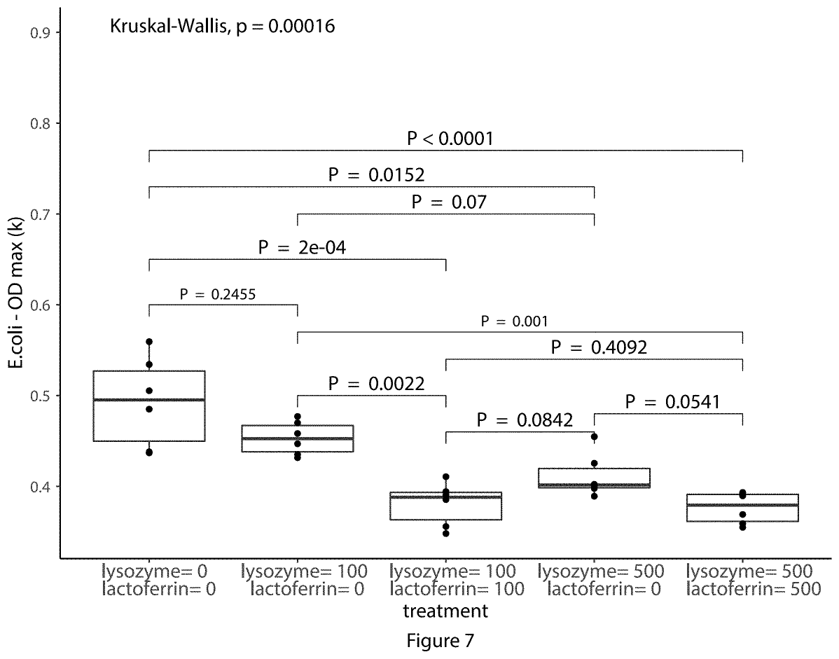

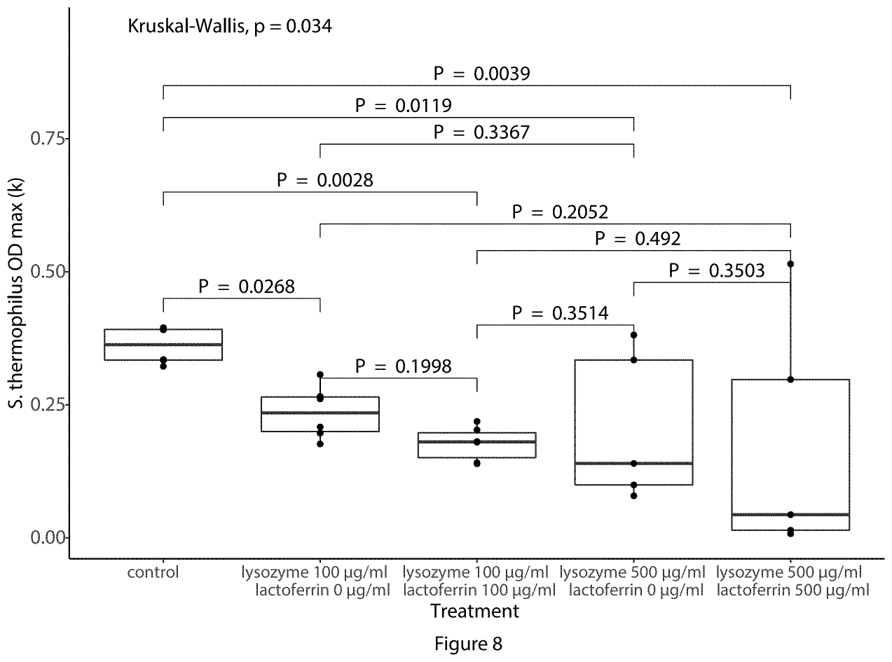

The tolerance of representative strains of taxa associated with dysbiosis (Table 3) to the combinatorial effect of lysozyme and lactoferrin was determined using the microdilution method. Overnight cultures were diluted 1:100 in fresh growth media. The bacterial solutions were mixed with an enzyme stock solutions with a final concentration of 100 μg/ml each of lysozyme and lactoferrin or with a final concentration of 500 μg/ml of each lactoferrin and lysozyme. The control solution was prepared by combining the bacterial solutions with Phosphate Buffered-Saline PBS. Mixtures were transferred to wells of a 96-well microplate. Microplates were incubated at 37° C. Bacterial growth was monitored every 30 minutes by optical density OD₆₀₀ over a time-course of twenty four hours. Data was generated for six biological replicates. Microbial growth kinetics were analyzed by plotting the optical density (OD₆₀₀) against time. The data was fitted to the logistic equation of bacterial growth, and highest bacterial growth density attained (OD max) was compared between enzyme treatments and the control. The combinatorial effect of lysozyme and lactoferrin at 100 μg/mL resulted in significantly lower OD max for E. coli compared to lysozyme alone 100 μg/mL (FIG. 7). Combining lysozyme and lactoferrin at 500 μg/mL resulted in significantly lower OD max (P<0.05; Kruskal-Wallis-Dunn's-adjusted) for E. coli compared to control but not to lysozyme alone at 500 μg/mL or both enzymes combined at 100 μg/mL. For S. thermophilus, all the concentrations of lysozyme tested resulted in significantly lower ODmax but the addition of lactoferrin did not result further significant growth reduction (FIG. 8). These results indicate that at certain concentrations the enzymes have a synergistic effect in reducing growth of gram negative taxa associated with dysbiosis such as E. coli and that lysozyme alone is sufficient to reduce growth of gram positive taxa associated with dysbiosis such as S. thermophilus.

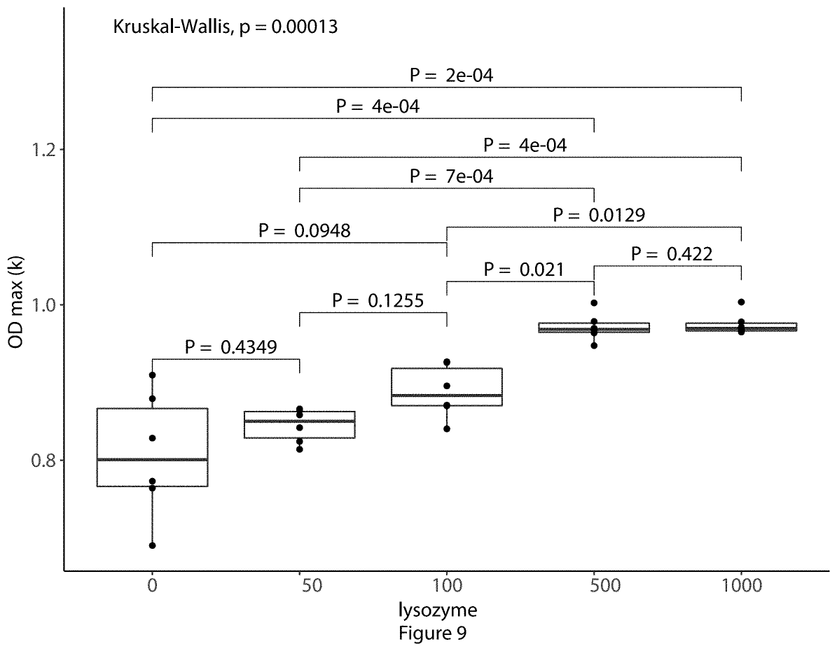

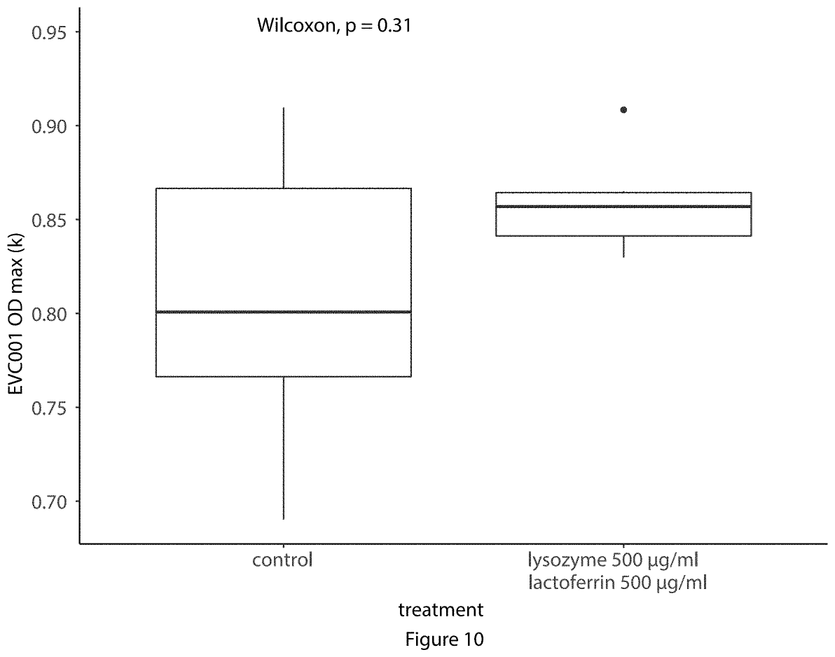

The effects of lysozyme and lactoferrin on the growth of B. infantis was determined using the microdilution method. Overnight cultures were diluted 1:100 in fresh growth media. The bacterial solutions were mixed with a enzyme stock solutions of lysozyme final concentration of 50, 100, 500 and 1000 μg/ml of lysozyme or with 500 μg/ml of each lactoferrin. The control solution was prepared by combining the bacterial solutions with Phosphate Buffered-Saline PBS. Mixtures were transferred to wells of a 96-well microplate. Microplates were incubated at 37° C. Bacterial growth was monitored every 30 minutes by optical density OD₆₀₀ over a time-course of forty eight hours. Data was generated for six biological replicates. Microbial growth kinetics were analyzed by plotting the optical density (OD₆₀₀) against time. The data was fitted to the logistic equation of bacterial growth, and highest bacterial growth density attained (OD max) was compared between enzyme treatments and the control. Unexpectedly, all concentrations of lysozyme tested resulted in a higher OD max for B. infantis compared to control indicating a positive relationship between increasing concentration of lysozyme and growth of B. infantis. Addition of 500 μg/mL resulted in significantly higher ODmax for B. infantis compared to control (P<0.05; Kruskal-Wallis-Dunn's-adjusted). Addition of 1000 μg/mL did not result in further growth (FIG. 9). The ODmax of B. infantis in the presence of both lactoferrin and lysozyme at 500 μg/mL each was not statistically different compared to control (P>0.05; Kruskal-Wallis-Dunn's-adjusted) (FIG. 10). These results suggest at certain concentrations the bioactive enzymes tested are favorable or have no effect on the B. infantis.

TABLE 3
Representative species of taxa associated with dysbiosis

| Species | Related diseases |
|---|---|
| Clostridium spp. | C. difficile diarrhea |
| Streptococcus spp. | Neonatal sepsis |
| Eschericha coli | Neonatal sepsis, NEC |
| Klebsiella spp. | Neonatal sepsis, NEC |

### Example 4. In Vitro Screening for Susceptibility of Dysbiotic Strains to Functional Bioactive Proteins in Conjunction with a Glycan

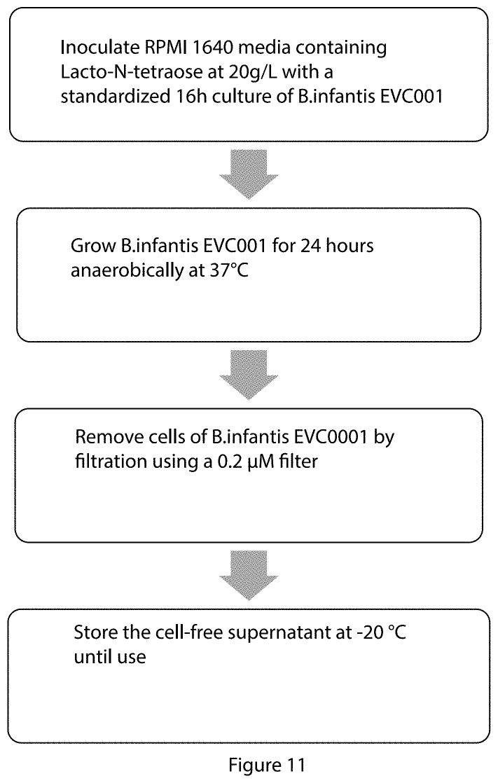

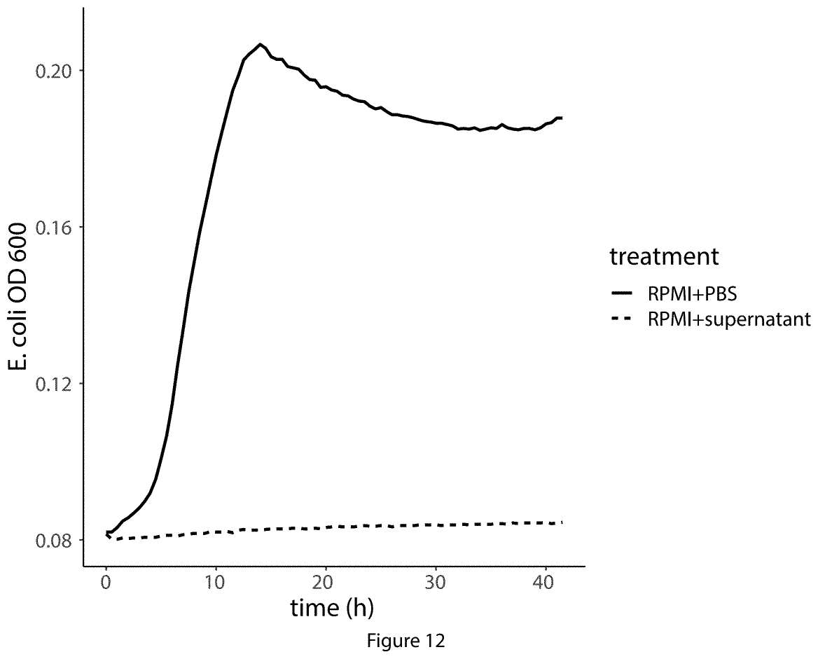

The bactericidal effects of a cell-free media in which B. infantis had previously grown in the presence of a glycan e.g. lacto-n-tetraose (LNT) were performed using the broth microdilution method. The supernatant was generated following the steps in the flow chart in FIG. 11. Overnight cultures of E. coli were diluted 1:100 in RPMI 1640 media. The bacterial solution was then mixed with the supernatant (treatment) or the same volume of PBS (control) in wells of a 96-well microplate. Microplates were incubated at 37° C. for 24 hours. Bacterial growth was monitored every 30 minutes by optical density OD₆₀₀ over twenty four hours. Data was generated for six biological replicates. Microbial growth kinetics were analyzed by plotting the optical density (OD₆₀₀) against time. No growth was observed in the cultures that contained the superman. This indicates the supernatant of B. infantis has a bacteriocidal effect on E. coli (FIG. 12).

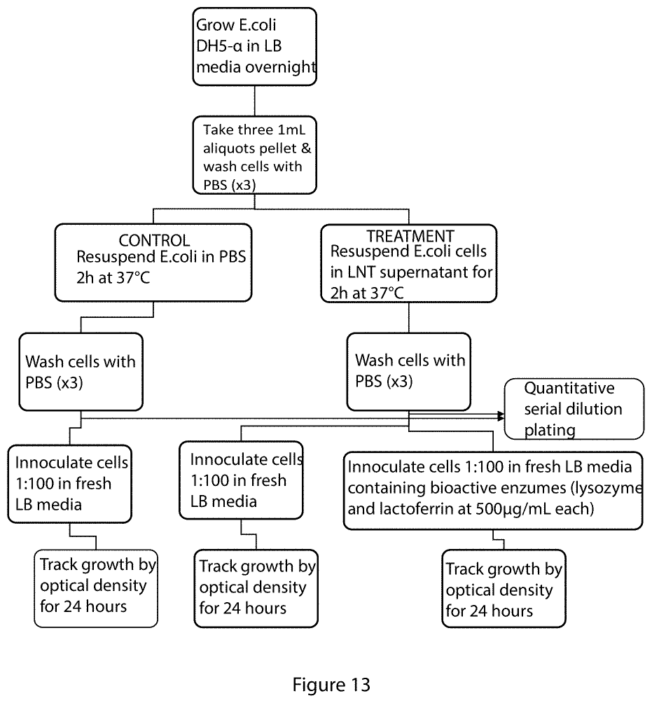

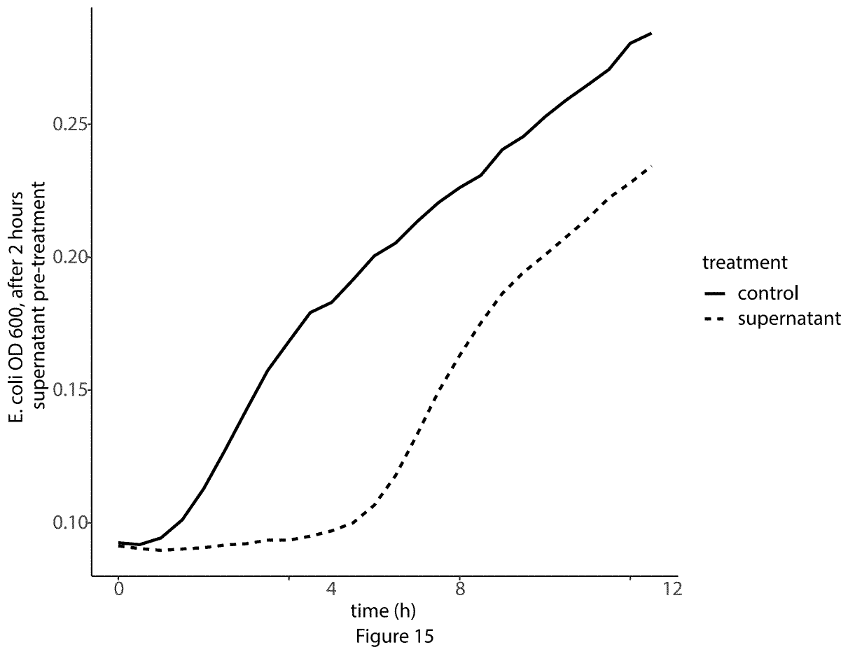

Bacteriocidal analyses were performed as described in FIG. 13 by treating cells of representative taxa associated with dysbiosis (Table 3) with cell-free media (supernatant) in which B. infantis had previously grown in the presence of a glycan e.g. lacto-n-tetraose (generated as described in FIG. 11). Bacterial cells from cultures of E. coli grown overnight were harvested by centrifugation and washed with an isotonic buffer (i.e Phosphate Buffered Saline; PBS). Cells were then resuspended in the supernatant (treatment) of PBS (control) and incubated for 2 hours at 37° C. The bactericidal effect of the supernatant is assessed by determining the viable cell numbers of bacterial cells (colony forming units; CFU) treated with the supernatant compared to those in the control by serial dilution plating. The number of viable cells of E. coli treated with the supernatant was on average 5.6 fold less compared to the number of viable cells in the control (1.97×10⁸ CFU/mL vs. 3.5×10⁷ CFU/mL) (FIG. 15). These results indicate the supernatant in which B. infantis had previously grown in the presence of LNT has bacteriocidal effects on taxa associated with dysbiosis such as E. coli.

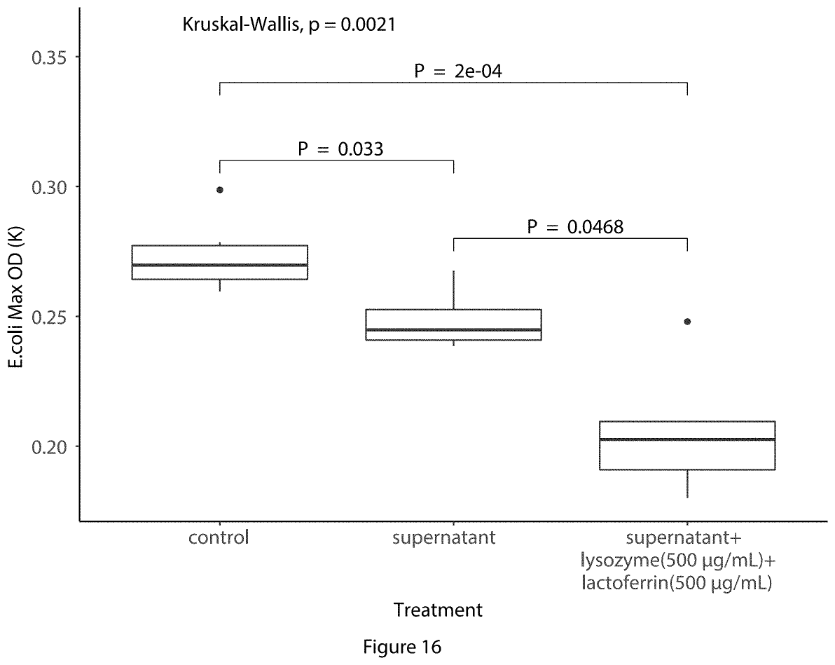

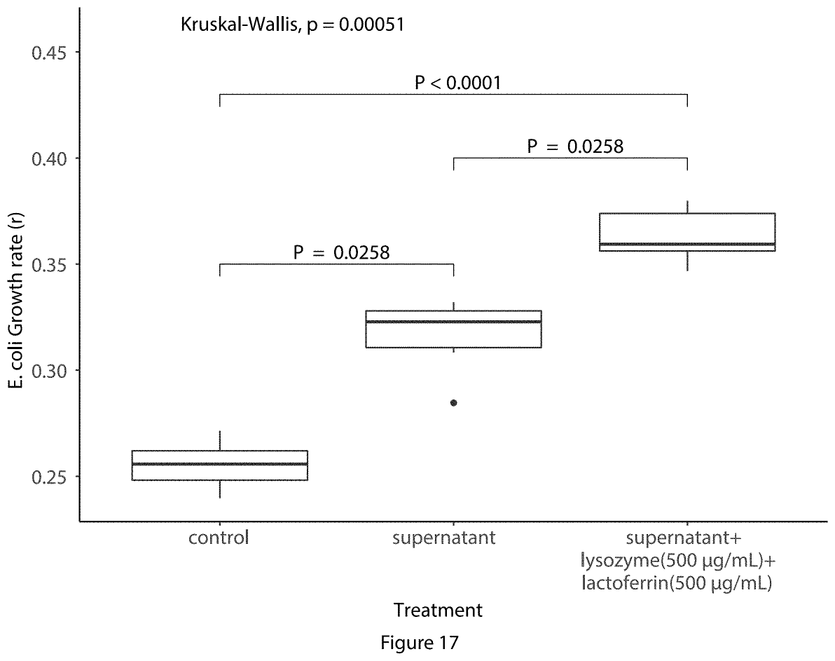

The susceptibility of dysbiotic strains to functional bioactive proteins in conjunction with a glycan supernatant were performed using the broth microdilution method. As described in the flow diagram in FIG. 13, bacterial cells of E. coli grown overnight were harvested by centrifugation and washed with an isotonic buffer (i.e Phosphate Buffered Saline; PBS). Cells were then resuspended in the supernatant (treatment) of PBS (control) and incubated for 2 hours at 37° C. Cells were then washed with PBS and diluted 1:100 in fresh liquid growth media. The bacterial solution was then mixed with solutions of lactoferrin and lysozyme at a final concentration of 500 μg/mL each (treatment) or 100 μL PBS (control) in wells of a 96-well microplate. Microplates were incubated at 37° C. for 12 hours. Bacterial growth was monitored every 30 minutes by optical density OD₆₀₀ over twenty four hours. Data was generated for six biological replicates. The data were fitted the logistic equation of microbial growth and parameters such as the length of the lag phase, time to reach exponential phase. ODmax and growth rate were compared between the treatments and the control. Cells treated with the supernatant had longer lag phases and attained a lower OD₆₀₀ on average by 12 hours which indicates a lower inoculum size. This resulted in significantly lower Max OD and slower growth rates compared to control (FIG. 16 and FIG. 17). Addition of the bioactive enzymes lysozyme and lactoferrin further reduced the growth, resulting in significantly lower OD max (FIG. 16) and significantly slower growth rates (FIG. 17 (P<0.05; Kruskal-Wallis-Dunn's-adjusted) compared to supernatant treatment alone. These results suggest a synergistic effect of supernant treatment and the bioactive enzymes to reduce growth of E. coli.

### Example 5. Preparation of an Infant Formula Comprising Lysozyme, Glycans, and/or EndoBI-1, and its Use with B. infantis

Preparation of an individual servings comprising lysozyme, HMO, and/or B. infantis. Sachets containing: 1) 0.0018 mg of lysozyme; 2) 0.0018 mg of lysozyme, plus 8 Billion cfu of B. infantis; and 3) 0.0018 mg of lysozyme, plus 8 Billion cfu of B. infantis, plus 0.709 grams LNT are prepared. The first sachet is added to 2 oz of a dried infant formula comprising HMO and B. infantis. The second sachet is added to 2 oz of a dried infant formula comprising. The third sachet is added to 2 oz of a dried infant formula. The appropriate number of sachets are blended with the amount of infant formula to be used, the mixture is reconstituted with water and fed to the infant in need of gut microbiome remodeling.

Preparation of an infant formula comprising glycans produced from EndoBI-1. The production of an infant formula is initiated by mixing of solids (powdered bovine milk or soy protein) and liquids (water and oil) in a high shear mixer under asceptic conditions. The enzyme EndoBI-1 is added to the mixture at a concentration of 10 mg/L. The temperature of the mixture is raised to 80 C for 30 min and them pumped through a plate and tube heat exchanger set at 140 C for rapid UHT sterilization. The mixture is then returned to 80 C, homogenized using controlled cavitation to increase spray drying efficiency and reduce fluid viscosity. The mixture is finally concentrated by evaporation under vacuum to a solids content of 20% and spray dried. The resulting powder is then dry blended with a vitamin/mineral premix to provide specifications compliant with the US Infant Formula Act of 1980. The resulting product comprises an inactivated EndoBI1 enzyme and the N-linked glycoproteins have been deglycosylated making the infant formula protein more digestible and hypoallergenic. The resulting product now also has free glycans that can feed B. infantis in the infant gut.

### Example 6. Addition of Endo-BI1 to a Nutritional Formula to Improve Digestibility and Reduce Allergenicity of Nutritional Material and Enhance Engraftment of B. infantis

The gene for EndoBI-1 is cloned and expressed without its membrane anchor sequence in E. coli and purified according to U.S. Pat. No. 9,327,016 (incorporated by reference). The purified enzyme is added directly to the nutritional formula slurry prior to spray drying at a concentration of 300 mg/L. The mixture is then spray dried and the resulting powder formula contains EndoBI-1 at a level of between 0.1 g/kg and 100 g/kg powder. Alternatively, the EndoBI-1 prepared in a sachet as in Example 1 can be added directly to the powder nutritional formula. A liquid mixture is then prepared by combining the powder nutritional formula with water at the temperature of 70 C. The EndoBI-1 immediately deglycoslates all N-linked glycoproteins rendering them more digestible and hypoallergenic and releasing glycans such as described in Example 4 for consumption by B. infantis. This nutritional drink is consumed by an individual who has finished a course of antibiotics or who is in need of gut microbiome rehabilitation.

### Example 7. The Effects of the Bioactive Proteins to in Modulating the Microbiome In Vivo can be Tested Using Humanized Mice

Briefly, germ free mice are “humanized” by oral gavage with a slurry of pooled human fecal samples from infants with dysbiotic microbiomes (i.e. high abundance of species with pathogenic and inflammatory potential and low abundance of bifidobacteria). Mice are also gavaged with an inoculum containing B. infantis EVC001. Mice are then separated in control and treatment groups. Control mice then fed an autoclaved standard diet. Treatment mice are fed custom diets containing combinations of oligosaccharides (e.g. LNT) and bioactive enzymes (e.g. lysozyme, lactoferrin EndoBI-1). Alternatively, treatment mice can be fed standard diets and oligosaccharides bioactive enzymes can be provided to the drinking water. Fecal samples are collected from the mice throughout the study, typically 5-10 days.

To determine the effect of the treatments, fecal bacterial numbers are determined by selective plating or taxa-specific qPCR. The composition of the fecal microbiota can be determined by 16s RNA gene or shotgun sequencing of DNA extracted from fecal samples. Results from these analyses are used to assess the effect of the treatments respective to control in the composition of the microbiome. Expected results are lower numbers of taxa with pathogenic potential and associated with dysbiosis (e.g. Enterobacteriaceae, Staphylococcaceae and Clostridiales) in mice receiving the treatment (B. infantis+combinations of oligosaccharide & bioactive enzymes) respective to control mice (B. infantis alone).

### Example 8. An Infant Formula Formulated to Include Lysozyme, Lactoferrin and LNT

The total protein is calculated such that the freeze dried functional bioactive proteins is included and provides at least 45 mg of each bioactive protein per 5 ounce serving dry blended into the formula. The LNT is included at at least 1.8 grams per 5 ounce serving. The remainder of the base formula is made to conform to infant formula regulations.

### Example 9. An Infant Formula Formulated to Include Lysozyme, Lactoferrin and DP2-3 FOS/GOS Mixture with Lactobacillus rhamnosus (LGG)

The total protein is calculated such that the freeze dried functional bioactive proteins is included and provides at least 45 mg of each bioactive protein per 5 ounce serving dry blended into the formula. The DP2-3 GOS/FOS is included at at least 0.9 grams per 5 ounce serving. The remainder of the base formula is made to conform to infant formula regulations.

1. A composition comprising a food preparation, a pharmaceutical preparation, or a dietary supplement wherein such food preparation, pharmaceutical preparation or dietary supplement comprises one or more functional bioactive protein(s) that are antimicrobial, improve digestibility of the food preparation, are a source of prebiotic glycans, are enzymes that can cleave glycans from glycoproteins, or that are responsible for pathogen deflection.

2. The composition of claim 1, wherein the functional bioactive protein is selected from glycoproteins, glycopeptides, protease or a lipase, an enzyme that can cleave N-linked or O-linked glycans from glycoproteins or glycopeptides, lactoferrin, lactoferricin, or lysozyme.

3. The composition of any preceding claim wherein the functional bioactive protein is lysozyme.

4. The composition of claim 3 wherein the lysozyme is present in a concentration less than 0.1 g/L.

5. The composition from claim 3 wherein the lysozyme is present in a concentration of 0.1-1.5 g/L.

6. The composition from claim 3 wherein the lysozyme is present in a concentration of 1.5 g/L to 3.1 g/L.

7. The composition of claim 3 wherein the lysozyme is present in a concentration of 3.1 g/L or greater.

8. The composition of claims 1-2 wherein the functional bioactive protein is lactoferrin.

9. The composition from claim 8 wherein the lactoferrin is present in a concentration of 0.1-10 g/L or greater.

10. The composition of claim 8 wherein the lactoferrin is present in a concentration of greater than 6 g/L.

11. The composition of claims 1-10, wherein the bioactive protein component is a combination of lysozyme and lactoferrin.

12. The composition of any one of claims 1-2 wherein the functional bioactive protein that can cleave N-glycans is an endo-β-N-acetylglucosaminidase.

13. The composition of claim 12 wherein the extracellular domain of recombinant Endo-β-N-acetylglucosaminidase is homologous to the Endo-β-N-acetylglucosaminidase found in B. infantis.

14. The composition of any one of claim 12-13, wherein the homologous amino acid sequence of the recombinant Endo-β-N-Acetylglucosaminidase is at least 80%, at least 85%, at least 90%, at least 95%, or at least 99% identical to the extracellular domain Endo-β-N-Acetylglucosaminidase found in B. infantis.

15. The composition of any one of claims 12-14, wherein the glycoprotein is contacted with Endo-β-N-acetylglucosaminidase and the composition comprises deglycosylated protein and released N-glycans.

16. The composition of claim 1-2 wherein the bioactive protein is a protease selected from trypsin and chymotrypsin.

17. The composition of claim 16 wherein the trypsin and/or chymotrypsin are present in a concentration of greater than 5.6 μg/L respectively.

18. The composition of claims 16-17 wherein the protease is added at a concentration of 0.01 g/kg to 5 g/kg.

19. The composition of claim 1-18 wherein the functional bioactive protein is a recombinant protein.

20. The composition of claim 19 wherein the recombinant protein is purified and/or dried.

21. The composition of any one of claims 1-20 wherein the composition further comprises one or more glycans with 2-8 sugar residues.

22. The composition of claim 21 wherein at least 1 monomer on the glycan is fucose or sialic acid residue.

23. The composition of claim 21-22 comprising a glycan that is identical to that from a mammalian milk oligosaccharide with a Type I core.

24. The composition of claim 21-23, where in the Type I core is LNT.

25. The composition of claim 21-22 comprising at a glycan that is identical to that from a mammalian milk oligosaccharide with a Type II core.

26. The composition of claim 25 wherein the Type II core is selected from LNnT.

27. The composition of any one of claims 21-26 comprising one or more glycans selected from the group: lacto-N-biose, N-acetyl lactosamine, lacto-N-triose, lacto-N-neotetrose, fucosyllactose, lacto-N fucopentose, lactodifucotetrose, sialyllactose, disialyllactone-N-tetrose, 2′-fucosyllactose, 3′-sialyllactosamine, 3′-fucosyllactose, 3′-sialyl-3-fucosyllactose, 3′-sialyllactose, 6′-sialyllactosamine, 6′-sialyllactose, difucosyllactose, lacto-N-fucosylpentose I, lacto-N-fucosylpentose II, lacto-N-fucosylpentose III, lacto-N-fucosylpentose V, sialyllacto-N-tetraose, derivatives thereof.

28. The composition of any one of claims 21-27, wherein the glycan is derived from a mammalian source.

29. The composition of claim 21-28, where the glycan is an N-glycan released from a glycoprotein.

30. The composition of claim 29 wherein the glycan purified from glycoproteins has at least one mannose residue.

31. The composition of claims 29-30, wherein the glycoprotein is from soy or whey.

32. The composition of claims 27-31, wherein a mammalian source of glycoprotein is supplemented with synthetically derived glycans.

33. The composition of claim 21-32, wherein the glycan is present in a concentration between 1-20 g/L.

34. The composition of claims 1-33, further comprising a Bifidobacterium species.

35. The composition of claim 34 wherein the Bifidobacterium species is selected from group consisting of B. longum subsp. longum, B. longum subsp. infantis (B. infantis), and B. breve.

36. The composition of claims 34-35 wherein the Bifidobacterium species selected expresses Endo-β-N-acetylglucosaminidase.

37. The composition of claims 35-36, wherein the Bifidobacterium species is B. infantis.

38. The composition of claims 34-37, wherein the B. infantis is activated.

39. The composition of claims 34-38 wherein the B. infantis is H5 competent.

40. The composition of claims 34-39, wherein the B. infantis is B. infantis EVC001.

41. The composition of claims 1-40, further comprising a Lactobacillus.

42. A composition comprising lysozyme and Bifidobacterium

43. The composition of claim 42 where the lysozyme is a recombinant lysozyme.

44. The composition of claims 42-43 wherein the Bifidobacterium is B. longum

45. The composition of claims 42-44 where the B. longum is B. longum subsp. infantis.

46. The composition of claims 42-45 where the B. longum subsp. infantis is activated.

47. The composition of claims 42-46 where the concentration of lysozyme is from 1-1,000 ug/ml.

48. The composition of claim 42-47 further comprising lactoferrin.

49. The composition of claim 48 where the lactoferrin is a recombinant lactoferrin.

50. The composition of claim 48-49 where the concentration of lactoferrin is from 1-1,000 ug/ml.

51. The composition of 41-50 further comprises a glycan.

52. The composition of any preceding claims, wherein a pharmaceutical composition is formulated as a unit dose medicament.

53. The composition of any of claim 1-52, wherein the food preparation is selected from the group consisting of infant formula, a milk replacer, an enteral nutrition product, and a meal replacer for a mammal.

54. The composition of any claims 1-52, wherein the dietary supplement is formulated as a capsule, sachet, lozenge, tablet, optionally an effervescent tablet, enema, suppository

55. The composition of any one of claims 1-52, wherein the composition is formulated as a capsule, packet, sachet, foodstuff, lozenge, tablet, optionally an effervescent tablet, enema, suppository, dry powder, dry powder suspended in an oil, chewable composition, syrup, or gel.

56. The composition of claims 1-52, wherein the composition is in the form of a dry powder or a dry powder suspended in an oil.

57. The composition of claims 1-52, wherein the functional bioactive protein and glycan are in an aqueous solution.

58. The composition of claim 57, wherein the aqueous solution is sterile.

59. The composition of any one of claims 1-58, wherein the selected bioactive protein or proteins is/are synthetically derived.

60. A method for reduction of pathogenic bacteria in the gut of a mammal, said method comprising administration of a composition of any one of claims 1-59 to reduce pathogenic bacteria.

61. The method of claim 60, wherein the pathogenic bacteria are selected from species of the genera: Escherichia, Klebsiella, Streptococcus and/or Clostridium.

62. The method of claims 60-61, wherein the mammalian subject's gut has been acidified following administration of the composition.

63. A method of improving the growth of Bifidobacterium in the gut of a mammal by administering the composition of claims 1-59.

64. The method of claim 60-63, wherein the administration of the composition leads to a 1-6 log increase in relative or absolute Bifidobacterium population in the subject's gut.

65. The method of claims 60-64, wherein the colonization results in a microbiome with greater than 60%, greater than 70%, greater than 80%, or greater than 90% of the total microbiome being Bifidobacterium.

66. The method of any of claims 60-65, wherein administering the composition referenced therein leads to improvement of growth rate of the mammal, said growth rate optionally expressed as Z scores, which may be selected from WAZ, LAZ, or WLZ.

67. A method comprising administering a composition of any of claims 1-57 to a mammal to treat or prevent autoimmune disorders, where the autoimmune disorder is selected from: Celiac disease, inflammatory bowel diseases (Crohn's, ulcerative colitis), Irritable Bowel Syndrome (IBS), Multiple sclerosis (MS), Type 1 diabetes mellitus, Psoriasis, atopic dermatitis, asthma, and/or food allergies.

68. A method comprising administering a composition of any of claims 1-57 to a mammal to treat or prevent disorders selected from: necrotizing enterocolitis, diaper rash, colic, late onset sepsis, colitis, gut pathogen overgrowth (e.g., C. difficile), hospital acquired infections, asthma, wheeze, allergic responses, obesity, Type II diabetes.

69. A method for recovery from chemotherapy or antibiotic treatment, the method comprising administering the composition of any one of claims 1-57 to a mammal contemporaneous with or following treatment.

70. A method for recovery from chemotherapy, surgery, antibiotic treatment, or gut dysbiosis, such method comprising administering the composition of any one of claims 1-57 to a mammal in conjunction with infant formula or a post-surgery recovery drink.

71. The method of any one of claims 60-70, wherein the mammal is a newborn, weaning, adult, geriatric mammal.

72. The method of claims 60-71 where the mammal is a human,

73. The method of claim 72 wherein the human is of 0-2 years of age.

74. The method of claim 72-73, wherein the human is a human infant.

75. The method of claim 72-74, wherein the human infant is fed with the compositions delivered in infant formula, human milk products, or human donor milk.

76. The method of any one of claims 60-71, wherein the mammal is a non-human mammal.

77. The method of claim 76, where in the mammal is a pig, horse, cow, dog, or cat.

78. The method of claim 60-71, wherein the composition is administered as an animal feed.


SEQUENCE LISTING (4 sequences)

SEQ ID NO: 1 - protein, 545 aa, Bifidobacterium infantis
MTFIKQMMPRYVASMTAGIVAAAMAATCAFAPVANADAVSPTQETIQSTGRHFMVYYRAWRDVTMKGVNTDLPDDNWISMYDIPYGVDVVNIFSYVPSGQEEQAQPFYDKLKSDYAPYLHSRGIKLVRGIDYTGVAVNGFRTFMKEQNKTESEATEADYDAYAKQVIDKYMISVGLDGLDIDMEAHPNDADVKISDNVIRALSKHIGPKSAKPDTTMFLYDTNGSYLNPFKNVAECFDYVAYQQYGSSSDRTARAAADYQPYIGNEFVPGLTFPEEGDMNNRWYDATEPYEESHFYQVASYVREHNLGGMFVYALDRDGRNYDEDLRRIVPSNLLWTKTAIAESEGMALDTAKTAANHYLDRMSLRQVIDDNAASADKARDMVGKAANLYETNKAVLGGDYGEGFSNTYDPTLEAGLLGIDISVLQQQIDKSSEIIGADTAESDAKTALRMARDAAIDGLTGKIYTADQVSAWSQALKAALDATVPVPTPDSTDQNGNRDKVTNHKVQGQPKQLSATGISTDIIVAVGVTLAIAGVALSLSRKLS

SEQ ID NO: 2 - protein, 555 aa, Bifidobacterium infantis
MCCSQGSEGRSSIGRRLAAVAAGVVATALMLGCSLAMGTVANAQEGESPVAASQEGNGNKHFMVYYRAWRDVTMKGVNTDLPDDNWISMYDIPYGIDVVNVFSYVPSGQEAAAQPFYDKLKSDYAPYLHARGIKLVRGLDYSGVMVDGFKTWIAQQGKNVDSATESDYDAYADHVIETYMTSVGLDGLDIDMETFPDAAQVAISDQVITALAKRIGPKSDNPEGTMFLYDTNGSYTAPFKNVSDCFDYVAYQQYGSDSNRTAKAAATYEQFIDSTKFVPGLTFPEEGDMNNRWNDATEPYLDSHFYDVASYSYDHNLGGMFVYALDRDGRTYSDDDLAHIKPSNLIWTKTAIAQSQGMSLENAKQAANHFLDRMSYTKDVPAETRQTVAAATNLYEVDKAVLGADWNDGYSNTYDPTLELSLTSIDTTALTGAIAKADALLADGATDTDVRTTLTTARNAAVTGLTSKLYTGADVVSWTASLNTAIADATGGKPKPQQPGTGETDKPSSGDASHNKPQSATGRLASTGSDGTVVLSVAVIMTLAGISVFAVRRRG

SEQ ID NO: 3 - protein, 483 aa, Bifidobacterium infantis
NADAVSPTQETIQSTGRHFMVYYRAWRDVTMKGVNTDLPDDNWISMYDIPYGVDVVNIFSYVPSGQEEQAQPFYDKLKSDYAPYLHSRGIKLVRGIDYTGVAVNGFRTFMKEQNKTESEATEADYDAYAKQVIDKYMISVGLDGLDIDMEAHPNDADVKISDNVIRALSKHIGPKSAKPDTTMFLYDTNGSYLNPFKNVAECFDYVAYQQYGSSSDRTARAAADYQPYIGNEFVPGLTFPEEGDMNNRWYDATEPYEESHFYQVASYVREHNLGGMFVYALDRDGRNYDEDLRRIVPSNLLWTKTAIAESEGMALDTAKTAANHYLDRMSLRQVIDDNAASADKARDMVGKAANLYETNKAVLGGDYGEGFSNTYDPTLEAGLLGIDISVLQQQIDKSSEIIGADTAESDAKTALRMARDAAIDGLTGKIYTADQVSAWSQALKAALDATVPVPTPDSTDQNGNRDKVTNHKVQGQPKQLSAT

SEQ ID NO: 4 - protein, 422 aa, Bifidobacterium infantis
VANAQEGDSPVAASQEGNGNKHFMVYYRAWRDVTMKGVNTDLPDDNWISMYDIPYGIDVVNVFSYVPSGQEAAAQPFYDKLKSDYAPYLHARGVKLVRGLDYSGVMVDGFKTWIAQQGKNVDSATESDYDAYADHVIETYMTSVGLDGLDIDMETFPDAAQVAISDQVITALAKRIGPKSDNPEGTMFLYDTNGSYTAPFKNVSDCFDYVAYQQYGSDSNRTAKAAATYEQFIDSTKFVPGLTFPEEGDMNNRWNDATEPYLDSHFYDVASYSYDHNLGGMFVYALDRDGRTYSDDDLAHIKPSNLIWTKTAIAQSQGMSLENAKQAANHFLDRMSYTKDVPAETRQTVAAATNLYEVNKAVLGADWNDGYSNTYDPTLELSLASIDTTALTGAIAKADALLADGATDTDVRTTLTTARNAA